SEQUENCE LISTING (25 sequences)

SEQ ID NO: 1 - protein, 15 aa, synthetic construct
  MOD_RES 1: note = biotinylated-alanine
AWLAYPDSVPYGSGC

SEQ ID NO: 2 - protein, 14 aa, synthetic construct
CGAWLAYPDSVPYR

SEQ ID NO: 3 - protein, 15 aa, synthetic construct
  MOD_RES 1: note = biotinylated-alanine
AWLAYPDSVPYRPKC

SEQ ID NO: 4 - protein, 16 aa, synthetic construct
CGAWLAYPDSVPYRPK

SEQ ID NO: 5 - protein, 16 aa, synthetic construct
  MOD_RES 16: note = amidated-Lysine
CGAWLAYPDSVPYRPK

SEQ ID NO: 6 - protein, 15 aa, synthetic construct
  MOD_RES 15: note = amidated-Lysine
GAWLAYPDSVPYRPK

SEQ ID NO: 7 - protein, 14 aa, synthetic construct
GAWLAYPDSVPYRP

SEQ ID NO: 8 - protein, 14 aa, synthetic construct
  MOD_RES 1: note = carbamidomethyl-cysteine
CGAWLAYPDSVPYR

SEQ ID NO: 9 - protein, 12 aa, synthetic construct
  VARIANT 1: note = Residue may be deleted
  VARIANT 12: note = Residue may be deleted
XWLAYPDSVPYX

SEQ ID NO: 10 - protein, 13 aa, synthetic construct
  VARIANT 1: note = A, biotinylated-alanine, C, azido-lysine, propargylglycine or deleted
  VARIANT 12..13: note = R, P-K, C or K wherein one position may be deleted
XWLAYPDSVPYXX

SEQ ID NO: 11 - protein, 14 aa, synthetic construct
  VARIANT 1..2: note = Y-S or W-L
  VARIANT 10..14: note = M-M-S, Amidated-Methionine, Amidated-Tyrosine, Y-K, Y-S-K, Y-G-S-K, Y-G-S-G-K, Y-R or Y-S wherein some positions may be deleted
XXAYPDSVPXXXXX

SEQ ID NO: 12 - protein, 4 aa, synthetic construct
YGSK

SEQ ID NO: 13 - protein, 5 aa, synthetic construct
YGSGK

SEQ ID NO: 14 - protein, 5 aa, synthetic construct
GSGSK

SEQ ID NO: 15 - protein, 4 aa, synthetic construct
YSAY

SEQ ID NO: 16 - protein, 7 aa, synthetic construct
AHHHHHH

SEQ ID NO: 17 - protein, 7 aa, synthetic construct
MASQGPG

SEQ ID NO: 18 - protein, 16 aa, synthetic construct
  MOD_RES 1: note = carbamidomethyl-cysteine
  MOD_RES 1: note = biotinylated-Lysine
CGAWLAYPDSVPYRPK

SEQ ID NO: 19 - protein, 16 aa, synthetic construct
  MOD_RES 16: note = Amidated-cysteine
GAWLAYPDSVPYRPKC

SEQ ID NO: 20 - protein, 15 aa, synthetic construct
  MOD_RES 1: note = azido-lysine
  MOD_RES 15: note = amidated-Lysine
KAWLAYPDSVPYRPK

SEQ ID NO: 21 - protein, 16 aa, synthetic construct
  MOD_RES 1: note = propargylglycine
  MOD_RES 16: note = amidated-Lysine
XGAWLAYPDSVPYRPK

SEQ ID NO: 22 - protein, 15 aa, synthetic construct
  MOD_RES 1: note = biotinylated-alanine
AWLAYPDSVPYRPKK

SEQ ID NO: 23 - protein, 14 aa, synthetic construct
  MOD_RES 1: note = biotinylated-alanine
AWLAYPDSVPYRPK

SEQ ID NO: 24 - protein, 17 aa, synthetic construct
  MOD_RES 17: note = biotinylated-Lysine
YSAYPDSVPMMSGSGSK

SEQ ID NO: 25 - protein, 15 aa, synthetic construct
  MOD_RES 1: note = biotinylated-alanine
AWLAYPDSVPYRPKG

(19) United States
(12) Patent Application Publication
(10) Pub. No.: US 2023/0391826 A1
(43) Pub. Date: Dec. 7, 2023

(54) COMPOSITIONS AND METHODS FOR TREATING DISEASES

(71) Applicant: Sanford Burnham Prebys Medical Discovery Institute, La Jolla, CA (US)

(72) Inventors: Elena B. PASQUALE, San Diego, CA (US); Bernhard C. LECHTENBERG, Melbourne (AU)

(21) Appl. No.: 18/172,116

(22) Filed: Aug. 28, 2023

Related U.S. Application Data
(63) Continuation of application No. PCT/US2021/050294, filed on Sep. 14, 2021.
(60) Provisional application No. 63/078,241, filed on Sep. 14, 2020.

(51) Int. Cl. C07K 7/08; A61P 35/00

(52) U.S. Cl. CPC C07K 7/08; A61P 35/00; A61K 38/00

(57) ABSTRACT

Disclosed herein are methods and compositions engineered to modulate ephrin type-A receptor 2 (EphA2), including novel compositions comprising one or more dimeric peptide units that binds to a EphA2, wherein the dimeric peptide comprises two or more homologous sequences or fragments thereof, wherein the two or more homologous sequences or fragments thereof, individually, comprise one or more binding sites for the EphA2 with unexpectedly high specificity and binding affinity. The compositions described herein can be attenuated to treat subjects suffering from diseases and/or conditions.

### CROSS-REFERENCE TO RELATED APPLICATIONS

This application is a continuation of International Application No. PCT/US2021/050294, filed on Sep. 14, 2021, which claims the benefit of U.S. Provisional Application No. 63/078,241, filed Sep. 14, 2020, all of which are hereby incorporated herein by reference in their entirety.

### STATEMENT AS TO FEDERALLY SPONSORED RESEARCH

This invention was made with government support under contract number R01 GM131374 and R01 NS087070 by the National Institutes of Health. The government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on Jun. 16, 2023, is named 42256-784_301_SL.xml and is 29,888 in size.

### BACKGROUND

EphA2 has been implicated in many disease processes. It is overexpressed in many cancer types where ligand-induced ephrin type-A receptor 2 (EphA2) kinase-dependent signaling is low (Barquilla and Pasquale, 2015; Miao and Wang, 2009; Pasquale, 2010). This apparent paradox can be explained by the fact that the receptor has pro-oncogenic activities in the absence of ligand. In contrast, EphA2 activation by ephrin-A ligands can inhibit oncogenic signaling networks (such as AKT-mTORC1 and RAS-ERK) and the pro-oncogenic EphA2 phosphorylation on 5897 and induce EphA2 internalization and degradation. Thus, agents promoting EphA2 activation are useful to suppress cancer cell malignancy as well as to deliver drugs, toxins and imaging agents to tumor cells. Additionally, inhibiting EphA2 activation is useful against pathological forms of angiogenesis, inflammation and parasitic infections.

### SUMMARY

Described herein are novel methods and compositions that are therapeutically effective in treating diseases, conditions, and/or subsets of diseases and conditions wherein the pathology of the disease, condition, or subset thereof involves the EphA2 receptor. EphA2 receptor activity contributes to many pathological conditions like cancer cell proliferation. Meanwhile, EphA2 receptor inhibition contributes to other pathological conditions like harmful inflammation and angiogenesis. The methods and compositions described herein provide novel techniques to modulate EphA2 activity to achieve a desired effect relative to the pathology experienced by the subject.

An aspect of this disclosure are compositions comprising one or more dimeric peptide units that binds to a ephrin type-A receptor 2 (EphA2), wherein the dimeric peptide comprises two or more homologous sequences or fragments thereof, wherein the two or more homologous sequences or fragments thereof, individually, comprise one or more binding sites for the EphA2.

In some embodiments, more than one of the homologous sequences simultaneously bind to EphA2. In some embodiments, more than one dimeric peptide unit simultaneously binds to EphA2. In some embodiments, more than one dimeric peptide units bind together to form an oligomer complex; wherein the oligomer complex binds to one or more binding sites on EphA2. In some embodiments, one or more of the homologous sequences comprise X1-Xaa1-Xaa2-Xaa3-Xaa4-Xaa5-Xaa6-Xaa7-Xaa8-Xaa9-Xaa10-X2,
wherein
X1 may be absent, or one or more amino acid selected from Table 1;
X2 may be absent, or one or more amino acid selected from Table 1;
Xaa1 may be absent, or W, Y, or F;
Xaa2 may be absent, or L or I;
Xaa3 may be absent, or A or V;
Xaa4 may be absent, or W, Y or F;
Xaa5 may be absent, or P;
Xaa6 may be absent, or D or E;
Xaa7 may be absent, or S or T;
Xaa8 may be absent, or A, V, or I;
Xaa9 may be absent, or P; and
Xaa10 may be absent, or W, Y, or F.

In some embodiments, X1 is absent, alanine (A), biotinylated-alanine (βA), a first spacer, cystine (C), azido-lysine (K_{N3}), propargylglycine (Pra), or any combination thereof. In some embodiments, X2 is absent, R, a second spacer, proline-lysine (P-K), C, K, or any combination thereof.

In some embodiments, one or more of the homologous sequences comprise X1-W-L-A-Y-P-D-S-V-P-Y-X2 (SEQ ID NO: 9), wherein
X1 is absent, or one or more amino acid selected from Table 1; and
   X2 is absent, or one or more amino acid selected from Table 1.

Another aspect of the present disclosure provides a composition comprising a peptide comprising at least a first subunit and a second subunit, wherein said first subunit comprises X1-W-L-A-Y-P-D-S-V-P-Y-X2 (SEQ ID NO: 9), and wherein said second subunit comprises X1-W-L-A-Y-P-D-S-V-P-Y-X2 (SEQ ID NO: 10), wherein X1 is A, βA, a first spacer, C, azido-lysine (K_{N3}), propargylglycine (Pra), or any combination thereof and X2 is R, a second spacer, P-K, C, K, or any combination thereof. In some embodiments, said C is carbamidomethyl-cysteine (C_{cam}). In some embodiments, a C-terminus of said first subset, said second subset, or both is amidated. In some embodiments, said first spacer and said second spacer comprise one or more amino acids. In some embodiments, said first spacer and said second spacer comprise a glycine. In some embodiments, said first spacer and said second spacer comprise a glycine and a serine. In some embodiments, said first subunit and said second subunit are homologous. In some embodiments, an N-terminus of said first subunit or said second subunit, or a C-terminus of said first subunit or said second subunit, or any combination thereof, comprises biotin. In some embodiments, said first subunit or said second subunit further comprises acetylation of a Lys14 side chain. In some embodiments, said composition comprises any one of the peptides listed in Table 2 or Table 3. A pharmaceutical composition comprising any one of the compositions of the embodiments disclosed herein; and one or more pharmaceutically acceptable excipients.

In some embodiments, the one or more homologous sequences comprise SEQ ID No. 1 (Table 2a.). In some embodiments, the one or more homologous sequences comprise SEQ ID No. 2 (Table 2a.). In some embodiments, the one or more homologous sequences comprise SEQ ID No. 3 (Table 2a.). In some embodiments, the one or more homologous sequences comprise SEQ ID No. 4 (Table 2a.). In some embodiments, the one or more homologous sequences comprise SEQ ID No. 5 (Table 2a.). In some embodiments, the one or more homologous sequences comprise SEQ ID No. 6 (Table 2a.). In some embodiments, the one or more sequences comprise SEQ ID No. 7 (Table 2a.). In some embodiments, the one or more homologous sequences comprise SEQ ID No. 8 (Table 2a.).

Another aspect of the present disclosure provides a method of treating a disease or condition in a subject in need thereof, the method comprising: administering the composition of any one the embodiments disclosed herein, or the pharmaceutical composition of any one of the embodiments disclosed herein, to said subject. In some embodiments, the method further comprises administering a half-life extending molecule to said subject. In some embodiments, said disease or condition is a parasitic infection. In some embodiments, said disease or condition is pathological forms of angiogenesis. In some embodiments, said disease or condition is an inflammatory disease. In some embodiments, said disease or condition is cancer. In some embodiments, said inflammatory disease is atherosclerosis, diabetes, arthritis, psoriasis, multiple sclerosis, lupus, inflammatory bowel disease, Addison's disease, Grave's disease, Sjogren's syndrome, Hashimoto's thyroiditis, Myasthenia gravis, Autoimmune vasculitis, Pernicious anemia, graft-versus-host disease, or Celiac disease. In some embodiments, said cancer is prostate cancer, castration resistant prostate cancer, neuroendocrine prostate cancer, transitional cell (or urothelial) prostate cancer, squamous cell prostate cancer, or small cell prostate cancer.

Another aspect of the present disclosure provides a method of preventing or reversing the onset of a subset of a disease or condition in a subject suffering from a disease or condition, the method comprising: administering the composition of any one of the embodiments disclosed herein, or the pharmaceutical composition of any one of the embodiments disclosed herein, to said subject. In some embodiments, the method further comprises administering a half-life extending molecule to said subject. In some embodiments, said disease or condition is a parasitic infection. In some embodiments, said disease or condition is pathological forms of angiogenesis. In some embodiments, said disease or condition is an inflammatory disease. In some embodiments, said disease or condition is cancer. In some embodiments, said inflammatory disease is atherosclerosis, diabetes, arthritis, psoriasis, multiple sclerosis, lupus, inflammatory bowel disease, Addison's disease, Grave's disease, Sjogren's syndrome, Hashimoto's thyroiditis, Myasthenia gravis, Autoimmune vasculitis, Pernicious anemia, graft-versus-host disease, or Celiac disease. In some embodiments, said cancer is prostate cancer, castration resistant prostate cancer, neuroendocrine prostate cancer, transitional cell (or urothelial) prostate cancer, squamous cell prostate cancer, or small cell prostate cancer.

In some aspects of the peptides and methods described herein, disclosed is a method of treating a disease or condition in a subject comprising administering to the subject a therapeutically effective amount of a peptide comprising X1-A-Y-P-D-S-V-P-X2 (SEQ ID NO: 11), wherein X1 is Y-S or W-L; X2 is any one of M-M-S, Mₐₘ, Yₐₘ, Y-K, Y-S-K, Y-G-S-K (SEQ ID NO: 12), Y-G-S-G-K (SEQ ID NO: 13), Y-R, or Y-S; and a half-life extending molecule, the addition of which slows down excretion of the peptide from the subject. In some embodiments, the peptide further comprises a GSGSK linker (SEQ ID NO: 14) on a carboxyl terminus (“C-terminal”). In some embodiments, the peptide further comprises biotin on the C-terminal. In some embodiments, the peptide further comprises a β-A (Alanine) on an amino terminus (“N-terminal”). In some embodiments, the peptide further comprises P-K on a carboxyl terminus (“C-terminal”). In some embodiments, the C-terminal of the peptide is amidated. In some embodiments, the peptide further comprises acetylation of a Lys14 side chain. In some embodiments, the peptide further comprises a biotinylated alanine on an amino terminus (“N-terminal”). In some embodiments, the peptide comprises any combination of further components described herein.

In another aspect, the methods disclosed herein comprise a method of treating a subtype of a disease or condition in a subject comprising administering to the subject a therapeutically effective amount of a peptide comprising X1-A-Y-P-D-S-V-P-X2 (SEQ ID NO: 11), wherein X1 is Y-S or W-L; X2 is any one of M-M-S, Mₐₘ, Yₐₘ, Y-K, Y-S-K, Y-G-S-K (SEQ ID NO: 12), Y-G-S-G-K (SEQ ID NO: 13), Y-R, or Y-S; and a half-life extending molecule, the addition of which slows down excretion of the peptide from the subject. In some embodiments, the peptide further comprises a GSGSK linker (SEQ ID NO: 14) on a carboxyl terminus (“C-terminal”). In some embodiments, the peptide further comprises biotin on the C-terminal. In some embodiments, the peptide further comprises a (3-A (Alanine) on an amino terminus (“N-terminal). In some embodiments, the peptide further comprises P-K on a carboxyl terminus (“C-terminal”). In some embodiments, the C-terminal of the peptide is amidated. In some embodiments, the peptide further comprises acetylation of a Lys14 side chain. In some embodiments, the peptide further comprises a biotinylated alanine on an amino terminus (“N-terminal”). In some embodiments, the peptide comprises any combination of further components described herein.

In another aspect, the methods disclosed herein comprise a method of preventing or reversing the onset of a subset of a disease or condition in a subject suffering from a disease or condition comprising administering to the subject a therapeutically effective amount of a peptide comprising X1-A-Y-P-D-S-V-P-X2 (SEQ ID NO: 11), wherein X1 is Y-S or W-L; X2 is any one of M-M-S, Mam, Yam, Y-K, Y-S-K, Y-G-S-K (SEQ ID NO: 12), Y-G-S-G-K (SEQ ID NO: 13), Y-R, or Y-S; and a half-life extending molecule, the addition of which slows down excretion of the peptide from the subject. In some embodiments, the peptide further comprises a GSGSK linker (SEQ ID NO: 14) on a carboxyl terminus (“C-terminal). In some embodiments, the peptide further comprises biotin on the C-terminal. In some embodiments, the peptide further comprises a β-A (Alanine) on an amino terminus (“N-terminal”). In some embodiments, the peptide further comprises P-K on a carboxyl terminus (“C-terminal”). In some embodiments, the C-terminal of the peptide is amidated. In some embodiments, the peptide further comprises acetylation of a Lys14 side chain. In some embodiments, the peptide further comprises a biotinylated alanine on an amino terminus (“N-terminal”). In some embodiments, the peptide comprises any combination of further components described herein. In some embodiments, the disease or condition is a parasitic infection. In some embodiments, the disease or condition is pathological forms of angiogenesis. In some embodiments, the disease or condition comprises an inflammatory disease. In some embodiments, the inflammatory disease is atherosclerosis. In some embodiments, the disease or condition is cancer. In some embodiments, the cancer comprises prostate cancer, castration resistant prostate cancer, neuroendocrine prostate cancer, transitional cell (or urothelial) prostate cancer, squamous cell prostate cancer, small cell prostate cancer, or a combination thereof.

In another aspect, the compositions disclosed herein comprise a composition comprising a peptide comprising X1-A-Y-P-D-S-V-P-X2 (SEQ ID NO: 11), wherein X1 is Y-S or W-L; and X2 is any one of M-M-S, Mam, Yam, Y-K, Y-S-K, Y-G-S-K (SEQ ID NO: 12), Y-G-S-G-K (SEQ ID NO: 13), Y-R, or Y-S. In some embodiments, the peptide further comprises a GSGSK linker (SEQ ID NO: 14) on a carboxyl terminus (“C-terminal”). In some embodiments, the peptide further comprises biotin on the C-terminal. In some embodiments, the peptide further comprises a β-A (Alanine) on an amino terminus (“N-terminal”). In some embodiments, the peptide further comprises P-K on a carboxyl terminus (“C-terminal”). In some embodiments, the C-terminal of the peptide is amidated. In some embodiments, the peptide further comprises acetylation of a Lys14 side chain. In some embodiments, the peptide further comprises a biotinylated alanine on an amino terminus (“N-terminal”). In some embodiments, the peptide comprises any combination of further components described herein.

In another aspect, the compositions disclosed herein comprise a composition comprising a peptide comprising X1-A-Y-P-D-S-V-P-X2 (SEQ ID NO: 11), wherein X1 is Y-S or W-L; X2 is any one of M-M-S, Mam, Yam, Y-K, Y-S-K, Y-G-S-K (SEQ ID NO: 12), Y-G-S-G-K (SEQ ID NO: 13), Y-R, or Y-S; and a half-life extending molecule, the addition of which slows down excretion of the peptide from a subject to which the peptide is administered. In some embodiments, the peptide further comprises a GSGSK linker (SEQ ID NO: 14) on a carboxyl terminus (“C-terminal”). In some embodiments, the peptide further comprises biotin on the C-terminal. In some embodiments, the peptide further comprises a β-A (Alanine) on an amino terminus (“N-terminal”). In some embodiments, the peptide further comprises P-K on a carboxyl terminus (“C-terminal”). In some embodiments, the C-terminal of the peptide is amidated. In some embodiments, the peptide further comprises acetylation of a Lys14 side chain. In some embodiments, the peptide further comprises a biotinylated alanine on an amino terminus (“N-terminal”). In some embodiments, the peptide comprises any combination of further components described herein. In some embodiments, the composition further comprises a carrier, such as a pharmaceutically acceptable carrier.

In another aspect, the methods disclosed herein comprise a method of preventing oligomerization of an EphA2 receptor comprising contacting the EphA2 receptor with a composition comprising a peptide comprising X1-A-Y-P-D-S-V-P-X2 (SEQ ID NO: 11), wherein X1 is Y-S or W-L; and X2 is any one of M-M-S, Mam, Yam, Y-K, Y-S-K, Y-G-S-K (SEQ ID NO: 12), Y-G-S-G-K (SEQ ID NO: 13), Y-R, or Y-S. In some embodiments, the peptide further comprises a GSGSK linker (SEQ ID NO: 14) on a carboxyl terminus (“C-terminal”). In some embodiments, the peptide further comprises biotin on the C-terminal. In some embodiments, the peptide further comprises a β-A (Alanine) on an amino terminus (“N-terminal”). In some embodiments, the peptide further comprises biotin on a carboxyl terminus (“C-terminal”). In some embodiments, the peptide further comprises P-K on a carboxyl terminus (“C-terminal”). In some embodiments, the C-terminal of the peptide is amidated. In some embodiments, the peptide further comprises acetylation of a Lys14 side chain. In some embodiments, the peptide further comprises a biotinylated alanine on an amino terminus (“N-terminal”). In some embodiments, the peptide comprises any combination of further components described herein. In some embodiments, the composition further comprises a half-life extending molecule, the addition of which slows down excretion of the peptide from a subject to which the peptide is administered.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A-1C illustrates an example of Potency and selectivity of EphA2-targeting dimeric peptides. (FIG. 1A) ELISAs comparing the ability of the peptides to inhibit binding of ephrin-A5 fused to alkaline phosphatase (ephrinA5-AP) to the immobilized EphA2 extracellular domain fused to the Fc portion of an antibody (EphA2-Fc). The graphs show averages ±SD from triplicate measurements from a representative experiment. IC₅₀ values calculated from the fitted curves in each experiment are shown. Averages of IC₅₀ values obtained from multiple experiments are shown in panel FIG. 1B and Table 3. The 10 nM peptide concentration is outlined in red. (FIG. 1B) Plot of the average IC₅₀ values (listed in Table 3) for different ligands. The graph shows averages, with error bars indicating SDs and dots indicating the individual measurements; a log scale is used for the Y axis. (FIG. 1C) EphrinA5-AP binding to EphA receptors and ephrinB2-AP binding to EphB receptors in the presence of dimeric peptides representing each of the three configurations. Values are normalized to ephrin binding without peptide. The graphs show averages and SDs from triplicate measurements (with each measurement shown as a dot). The peptides were used at a concentration corresponding to ˜100-fold their IC₅₀ value: 65 nM for dimer (2), 60 nM for dimer (5) and 40 nM for dimer (8).

FIG. 2A-2E illustrates an example where dimeric peptides efficiently promote EphA2 autophosphorylation and downstream signaling. (FIG. 2A) Dose-response curves for EphA2 autophosphorylation on tyrosine 588 (pY588; purple) and for downstream inhibition of AKT phosphorylation (magenta). PC3 cells were treated for 15 min with different concentrations of the indicated peptides. EphA2 pY588 (indicative of receptor activation), total EphA2, AKT phosphorylation on S473 (pAKT, indicative of AKT activation) and total AKT were quantified from immunoblots. pY588/EphA2 values were normalized to the value obtained with saturating ephrinA1-Fc concentration. pAKT inhibition was calculated as 1-pAKT/AKT values normalized to the level in cells not treated with ligand. The graphs show quantifications from multiple blots (averages ±SE; the number of experiments used to generate each curve is shown in Table 3). EC₅₀ values (nM, shown) were calculated by non-linear regression with a Hillslope of 1 for the peptides and of 2 for ephrinA1-Fc and m-ephrinA1; the 10 nM concentration is outlined in red. (FIG. 2B) Examples of immunoblots of lysates from PC3 cells treated with the indicated concentrations of representative ligands. Y indicates treatment with 100 μM of the previously identified YSA-GSGSK-bio monomer (2*), which was included in all blots for comparison. A white vertical line indicates removal of irrelevant lanes. (FIG. 2C) The highest EphA2 Y588 phosphorylation induced by each ligand (Eₜₒₚ pY588) depends on the type of ligand and dimeric configuration. The graphs show the Y588/EphA2 values induced by 15 min stimulation with saturating concentrations of the different ligands normalized to the value for the reference ligand ephrinA1-Fc. The bars show averages ±SE, and the individual measurements are shown as black dots. The asterisks indicate the significance of the difference from ephrinA1-Fc, calculated using one-way ANOVA followed by the Dunnett's multiple comparisons test (**, P<0.01; ****, P<0.0001; ns, not significant). (FIG. 2D, FIG. 2E) Different ligands cause different Eₜₒₚ pY588, EC₅₀ pY588 and EC₅₀ pAKT inhibition (inh) but similar Eₜₒₚ pAKT inh. Plots of Eₜₒₚ versus EC₅₀ for pY588 in FIG. 2D and for pAKT inh in FIG. 2E. Averages ±SE are shown for the peptides and ephrinA1.

FIG. 3A-3E illustrates an example where different EphA2 ligands regulate pY588 phosphorylation and AKT inhibition with distinct kinetics. PC3 cells were treated for the indicated time periods with saturating concentrations of ephrinA1-Fc, dimeric peptides representative of each configuration, or monomeric peptide (10). Y588 and AKT phosphorylation levels and total EphA2 and AKT levels were quantified from immunoblots of cell lysates. (FIG. 3A) pY588/EphA2, normalized to the peak value. (FIG. 3B) EphA2/AKT (with AKT used as loading control) normalized to the average of the values at 0, 2.5, 5 and 10 min (when receptor degradation does not yet occur). (FIG. 3C) pY588/AKT, normalized to the peak value. (FIG. 3D) pAKT/AKT, normalized to the “0” time point corresponding to no ligand treatment. (FIG. 3E) AKT values normalized to the average of all the values for each ligand. The graphs show averages ±SE from 3 to 8 independent measurements. The asterisks indicate the significance of the difference from ephrinA1-Fc for the last 3 time points, calculated by mixed-effects analysis followed by the Dunnett's multiple comparisons test (*, P<0.05; **, P<0.01; ***, P<0.001; ****, P<0.0001; P=0.051 is also indicated, with the color of the asterisks indicating which peptide is significantly different from ephrinA1-Fc).

FIG. 4A-4E illustrates an example where dimers with different configurations induce EphA2 oligomerization and patching on the cell surface. (FIG. 4A-4D) Oligomerization curves comparing EphA2 WT and the G131Y and L223R/L254R/V255 interface mutants transiently expressed in HEK293 cells and treated with saturating concentrations of C-terminally linked dimer (2) or N-terminally linked dimer (5). The curves were obtained by fitting quantitative FRET data to monomer-oligomer models. Curves derived from best fit monomer-dimer models are shown as solid lines, and curves derived from best fit monomer-higher order oligomer models are shown as dashed lines. Curves for higher order oligomerization are steeper than dimerization curves. (FIG. 4E) Two-photon integrated fluorescence images of HEK293 cells expressing EphA2-EYFP and EphA2-mTURQ and under hypo-osmotic conditions. The plasma membrane regions in the rectangular yellow outlines are enlarged in the insets at the bottom of each panel. The dimeric peptides induce patching of EphA2 WT on the plasma membrane and dimer (5) also induces patching of the EphA2 L223R/L254R/V255R mutant, while in the other cases the EphA2 interface mutations disrupt patching. The scale bar represents 10 μm for the main panels and 4 μm for the insets. # indicates the presence of EphA2 patches in the plasma membrane.

FIG. 5A-5D illustrates an example where a flexible juxtamembrane segment is required for EphA2 autophosphorylation. HEK293 cells stably transfected with EGFP as a control, EphA2 WT or the EphA2 juxtamembrane deletion mutants ΔQ565-L582 (Δjxtm-1) and ΔQ565-T606 (Δjxtm-2) were treated for 2.5 min with saturating concentrations of (FIG. 5A) ephrinA1-Fc, (FIG. 5B) dimer (2), (FIG. 5C) dimer (6) and (FIG. 5D) dimer (8). Lysates were probed by immunoblotting with the indicated antibodies. The vertical line in FIG. 5B and FIG. 5D indicates removal of irrelevant lanes. The graphs show quantifications normalized for each antibody to the cells expressing EphA2 WT and not treated with ligand (lighter bars). In the case of EphA2 phosphorylation, the background from control lanes was subtracted before normalizing to EphA2 levels. The error bars represent SEs and the individual measurements from 3 experiments are shown as dots.

FIG. 6 illustrates an example where Ligands can bias EphA2 signaling responses. The bias factor β_{lig} was calculated for the indicated ligands using ephrinA1-Fc as the reference ligand. The error bars represent SEs and the number of experiments is indicated in Table 3. Statistical significance for the comparison of the different ligands with the reference ligand ephrinA1-Fc (β_{lig}=0) was determined by one sample t test; *, P<0.05; **, P<0.01; ***, P<0.001; ****, P<0.0001.

FIG. 7 illustrates an example of EphA2 domain structure, oligomerization and regulation of AKT signaling. Schematic representation of four EphA2 receptors oligomerized in the plasma membrane through the dimerization and the clustering interfaces. The ephrin-binding pocket, ATP-binding pocket and the different domains are labeled. Four tyrosine phosphorylation sites characteristic of the activated receptor are shown as orange circles. Phosphorylated S473 in AKT is shown as a red circle. SH2, SH2 domain-containing protein binding to the Y588 and/or Y594 phosphorylated motifs; Eff., downstream effector; PP1, Protein phosphatase 1-like phosphatase; PI3K, phosphatidylinositol 3-kinase.

FIG. 8A-8D illustrates an example of structural models of EphA2 LBD dimers induced by dimeric peptide ligands with different configurations. (FIG. 8A) Model of an EphA2 LBD dimer bound to the C-terminally-linked βA-WLA-YGSGC dimer (1). The two EphA2 LBDs are shown in grey surface with the peptide in light blue sticks and the disulfide bond in yellow. EphA2 Tyr48 is transparent to show the disulfide linking the two peptide moieties underneath. The middle panel shows a rotated view, with the C-termini of the EphA2 LBDs indicated in red. The right panel shows a schematic of the left panel, with the two monomeric moieties of each dimeric peptide shown as lines, with blue dots marking their N-termini (N-term) and red dots marking their C-termini (C-term) to illustrate the different configurations. A schematic of the peptide is also shown under the peptide name. (FIG. 8B) EphA2 LBDs in complex with the two monomeric precursors of the N-terminally-linked CGA-WLA-YRPK dimer (5) in orange sticks. In this arrangement of the LBDs, the N-termini of the two peptides are ˜15 Å apart. (FIG. 8C) Manual model based on the model in panel FIG. 8B and generated by moving the EphA2 LBDs together to bring the N-termini of the two monomeric precursors in close enough proximity for disulfide bond formation. (FIG. 8D) EphA2 LBDs in complex with head-to-tail dimer (8) (purple). The models are based on our previous crystal structure of the EphA2 LBD in complex with the βA-WLA-YRPK-bio (17*) peptide (PDB 6NK1). DE, DE loop; GH, GH loop; JK, JK loop; the two EphA2 molecules, mol A and mol B, are indicated.

FIG. 9 illustrates an example of EphA2 LBD interaction surfaces in different dimers. The EphA2 LBD is colored based on the residues participating in the different dimeric interfaces shown in FIG. 8A-8D. The modeled dimer interfaces induced by dimeric peptides (1) and (8) share some of the same residues, whereas the modeled dimer interface induced by dimer (5) in FIG. 8C involves different residues located on a different side of the LBD. The common peptide core (Trp2-Tyr11) is shown as yellow sticks.

FIG. 10A-10E illustrates an example of Representative ITC traces for the binding of dimeric peptides to the EphA2 LBD. (FIG. 10A-10E) The EphA2 LBD was titrated at 200-250 μM into 10-12.5 μM of the indicated peptide. The binding stoichiometry (N) and affinity (K_{d}) calculated from the titrations shown are indicated, while Table 3 reports average values from different experiments.

FIG. 11A-11D illustrates an example of different EphA2 ligands regulate pY588 phosphorylation and AKT inhibition with distinct kinetics. Same data as in FIG. 3A-3E, but with each curve for each peptide shown separately. (FIG. 11A) pY588/EphA2, normalized to the peak value. (FIG. 11B) EphA2/AKT (with AKT used as loading control) normalized to the average of the values at 0, 2.5, 5 and 10 min (when receptor degradation does not yet occur). (FIG. 11C) pY588/AKT, normalized to the peak value. (FIG. 11D) pAKT/AKT, normalized to the “0” time point corresponding to no ligand treatment. The graphs show averages ±SE from 3 to 8 independent measurements and percentage values are shown at 15 min, 1 hour and/or 3 hours.

FIG. 12 illustrates an example of FRET efficiencies versus total receptor concentrations. HEK293T cells were co-transfected with cDNAs encoding EphA2-mTURQ (donor) and EphA2-EYFP (acceptor). EphA2 was either wild-type (WT), the G131Y dimerization interface mutant, or the L223R/L254R/V255R clustering interface mutant. The cells were left untreated or stimulated with saturating concentrations of the indicated dimeric peptides. Individual data points are shown, each obtained from a different plasma membrane region, and their number is indicated in the graphs. Colors are the same as in FIG. 4A-4E.

FIG. 13 illustrates an example of PI3 kinase mediates basal and EphA2-dependent AKT activation in HEK293 cells. Lysates from HEK293 cells expressing EphA2 WT were treated with vehicle control (−) or with the PI3 kinase inhibitor LY294002 (+) and then stimulated for 5 min with dimer (2) or dimer (8) and probed with the indicated antibodies.

FIG. 14A-14E illustrates an example where distinct factors are responsible for EphA2 biased signaling induced by different ligands. (FIG. 14A) Eₜₒₚ values for EphA2 Y588 phosphorylation, as in FIG. 2C. (FIG. 14B) Eₜₒₚ values for inhibition of AKT phosphorylation. (FIG. 14C) Graph of the ratios of the Eₜₒₚ values for EphA2 Y588 phosphorylation and inhibition of AKT phosphorylation, which are used in the β_{lig} calculation. (FIG. 14D) Graph of the ratios of the EC₅₀ values for EphA2 Y588 phosphorylation and inhibition of AKT phosphorylation, which are used in the β_{lig} calculation. (FIG. 14E) Equation used to calculate the bias factor β_{lig} for each ligand. The terms of the equation are rearranged, compared to equation (1) in the Materials and Methods, to better conform to the graphs in FIG. 14A-14D. **, P<0.01; ***, P<0.001; ****, P<0.0001; ns, not significant for the comparison with ephrinA1-Fc by one-way ANOVA and Dunnett's posthoc test. The number of experiments used to calculate the different parameters is shown in Table 3.

### DETAILED DESCRIPTION OF THE INVENTION

The EphA2 receptor tyrosine kinase plays an important role in a plethora of biological and disease processes, ranging from angiogenesis and cancer to inflammation and parasitic infections. EphA2 is therefore considered an important drug target. Efforts to target EphA2 and modulate its activation and downstream signaling have included different strategies. The ATP binding site in the kinase domain is suitable for targeting with small molecule inhibitors, but it is difficult to achieve specific targeting given the high conservation of this site in Eph receptors and other kinases (Barquilla and Pasquale, 2015; Boyd et al., 2014; Noberini et al., 2012). The ephrin-binding pocket in the ligand binding domain (“LBD”) can also be targeted with engineered forms of the ephrin-A ligands, but these ligands bind promiscuously to all nine EphA receptors and are therefore not well-suited as selective EphA2 modulators (Barquilla and Pasquale, 2015; Boyd et al., 2014; Pasquale, 2010). The ephrin-binding pocket has also proven too large for selective high-affinity binding of small molecules (Barquilla and Pasquale, 2015; Noberini et al., 2012).

Prior to the methods and compositions described herein, the peptides known to bind to Eph receptors generally exhibited low binding affinity and low potency. Described herein are peptides which target the ephrin-binding pocket of EphA2 specifically, and mimic the binding features of the ephrin-A ligands. The peptides described herein comprise improvements including, but not limited to, low nanomolar potency.

Further, the peptides described herein comprise modifications including, but not limited to, carboxyl-terminus (“C-terminal”) modifications that convert peptide derivatives from antagonists to agonists that bridge two EphA2 molecules to promote receptor autophosphorylation and downstream signaling. Also described herein are features conferring agonistic or antagonistic properties, which can be useful for different applications, and show that the peptide agonists promote EphA2 oligomerization through an unexpected bivalent binding mode.

The following definitions should be used for amino acid abbreviations described herein:

TABLE 1

| Abbreviation | 1 letter abbreviation | Amino acid name |
|---|---|---|
| Ala | A | Alanine |
| Arg | R | Arginine |
| Asn | N | Asparagine |
| Asp | D | Aspartic acid |
| Cys | C | Cysteine |
| Gln | Q | Glutamine |
| Glu | E | Glutamic acid |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Pyl | O | Pyrrolysine |
| Ser | S | Serine |
| Sec | U | Selenocysteine |
| Thr | T | Threonine |
| Trp | W | Tryptophan |
| Tyr | Y | Tyrosine |
| Val | V | Valine |
| Asx | B | Aspartic acid or Asparagine |
| Glx | Z | Glutamic acid or Glutamine |
| Xaa | X | Any amino acid |
| Xle | J | Leucine or Isoleucine |

For convenience, reference to a specific amino acid involved in a linkage can use the nomenclature for the unlinked amino acid (e.g., the structure it may have prior to formation of a linkage). It is also understood that certain linkages, e.g., synthetic linkages, may not be formed by connecting two amino acids or derivatives as commonly referenced in the art. Therefore, references to linked amino acids herein may use the most closely approximating language to describe each involved chemical entity at a given residue position in the peptide antagonist. Correspondingly, linked entities in the peptide sequence, e.g., Xaa3, Xaa4, Xaa8, and Xaa14, may be referred to as linked amino acids, although they are not amino acids as commonly referenced in the art. In some embodiments, Xaa3 and Xaa8, and Xaa4 and Xaa14, when linked entities (e.g., forming an Xaa3-Xaa8 linkage and an Xaa4-Xaa14 linkage), can be referred to as linked (or linkage-forming) amino acids, linked (or linkage-forming) amino acid derivatives, linked (or linkage-forming) molecules, linked (or linkage-forming) moieties, linked (or linkage-forming) residues, or linked (or linkage-forming) entities in the alternative. These terms can be used to refer to amino acids, molecules, moieties, residues, or entities present at any of Xaa3, Xaa4, Xaa8, or Xaa14, in the alternative, either when linked or unlinked. For example, when not linked but intended to be linked in a peptide antagonist of the present disclosure, two linkage amino acids also can be referred to as linked (or linkage-forming) amino acids, linked (or linkage-forming) amino acid derivatives, linked (or linkage-forming) molecules, linked (or linkage-forming) moieties, linked (or linkage-forming) residues, or linked (or linkage-forming) entities in the alternative. When linked, two linkage amino acids can be referred to as linked (or linkage-forming) amino acids, linked (or linkage-forming) amino acid derivatives, linked (or linkage-forming) molecules, linked (or linkage-forming) moieties, linked (or linkage-forming) residues, or linked (or linkage-forming) entities, in the alternative. When not linked and not intended to be linked, two amino acids can be referred to as unlinked (or non-linkage forming) amino acids, unlinked (or non-linkage forming) amino acid derivatives, unlinked molecules, unlinked moieties, unlinked residues, or unlinked entities. In some embodiments, each residue at a non-linked amino acid position in a peptide antagonist of the present disclosure can be referred to as an amino acid, amino acid derivative, molecule, moiety, residue or entity, or as an unlinked (or non-linkage forming) amino acid, unlinked (or non-linkage forming) amino acid derivative, unlinked (or non-linkage forming) molecule, unlinked (or non-linkage forming) moiety, unlinked (or non-linkage forming) residue or unlinked (or non-linkage forming) entity.

Any constraining structure known to those of skill in the art is contemplated for linking the residues. Examples of constraining structures and their respective linkage residues include, but are not limited to linkages or bridges selected from: a disulfide bridge (e.g., a Cys-Cys linkage, wherein each linkage amino acid is a Cys); a Sec-Sec linkage (selenocysteine linkage, wherein each linkage amino acid is a selenocysteine); a cystathionine linkage or bridge (e.g., Ser-Homocysteine linkage), also referred to herein as Cyt-Cyt (e.g., CH₂—CH₂—S—CH₂); a lactam bridge (e.g., Asp-Lys or Glu-Lys linkage), a thioether linkage (e.g., a lanthionine linkage, including but not limited to Cys—dehydroalanine or methyl variant), and a dicarba linkage (e.g., a linkage of an olefin-containing amino acid, e.g., allyl glycine or prenyl glycine). In some embodiments, a linkage is selected from: a disulfide bridge having linkage residues Cys-Cys; a selenocysteine linkage having linkage residues Sec-Sec; a cystathionine linkage having linkage residues Ser-Homocysteine; a lactam bridge having residues Asp-Lys or Glu-Lys; a lanthionine linkage having linkage residues Cys—dehydroalanine or a methyl variant, and a dicarba linkage having linkage residues allyl glycine or prenyl glycine. In embodiments, linkage amino acid, linkage amino acid derivative, linkage molecule, linkage moiety, linkage residue, or linkage entity is selected from Cys, Sec, Ser, Homocysteine, Asp, Lys, Glu, dehydroalanine, or an olefin containing amino acid (e.g., allyl glycine or prenyl glycine).

### Novel Peptides

Described herein are engineered nanomolar peptide agonists as well as antagonists that target the ephrin-binding pocket of the EphA2 receptor tyrosine kinase by using as the starting point two peptides with high specificity for EphA2 but modest (micromolar) binding affinity. Improvements guided by structural information obtained from four different peptides crystallized in complex with the EphA2 LBD have resulted in up to a surprising 350-fold increase in binding affinity. Even more surprisingly, is that this vast improvement in binding affinity was achieved with only small changes in the size of the optimized peptide agonists and antagonists. The sequences for exemplary peptides described herein can be found in Table 2.

The extensive network of interactions with EphA2 involving almost all the residues of A-WLA-YRPK, which is documented in the crystal structure of the peptide in complex with the EphA2 LBD, is consistent with the potency improvements observed with each additional amino acid modification in the series of engineered peptides. Interestingly, the binding of the YSA derivatives analyzed by isothermal titration calorimetry (ITC) was characterized by unusually large decreases of both entropy and enthalpy. This might be expected for linear peptides that are unstructured and highly flexible in solution (resulting in an unfavorable decrease in entropy upon binding EphA2) but in which many of the residues contribute to the binding interaction with the receptor (resulting in a favorable decrease in enthalpy). The enthalpy component predominates in the best peptides that were developed, which exhibit low nanomolar affinity for EphA2. They therefore represent a marked improvement over the original peptides and their derivatives of similarly low potency that have been used by many groups over the years (Riedl and Pasquale, 2015).

TABLE 2a
Homologous sequences employed in novel
dimeric peptides

| SEQ ID NO. | SEQUENCE |
|---|---|
| 1 | βAWLAYPDSVPYGSGC |
| 2 | CGAWLAYPDSVPYR |
| 3 | βAWLAYPDSVPYRPKC |
| 4 | CGAWLAYPDSVPYRPK |
| 5 | CGAWLAYPDSVPYRPKₐₘ |
| 6 | GAWLAYPDSVPYRPKₐₘ |
| 7 | GAWLAYPDSVPYRP |
| 8 | C_{cam}GAWLAYPDSVPYR |

### Mechanism of Biotin in the Peptides Described Herein

Dimeric peptides can function as EphA2 agonists and Eph receptor activation is known to require oligomerization. Surprisingly, disclosed herein it is established that a C-terminal biotin confers the ability to efficiently promote EphA2 activation and downstream signaling in cells. Several pieces of evidence show that the likely explanation for the agonistic activity of the biotinylated peptide derivatives disclosed herein is that they function as bivalent ligands capable of bridging two EphA2 molecules. The X-ray crystal structures show distinct binding sites in the EphA2 LBD for the peptide N-terminal residues and the biotin, but do not conclusively show whether a peptide binds to two different EphA2 LBD molecules or to two binding sites within the same molecule, because of the lack of definition of the connecting residues. Nevertheless, the orientation of the biotin suggested by the shape of its electron density strongly suggests its interaction with a second EphA2 LBD molecule.

EphA2 Targeting Peptides
Dimeric peptides targeting EphA2

| Identi- | | SEQ | | | | |
|---|---|---|---|---|---|---|
| fier | | ID | | Inc₅₀ (nM) | K_{D} (nM) by | Eₘₐₓ |
| number | Name | NO | Sequence | by ELISA | ITC³ | PY588⁴ |
| 1 | B-WLA-YGSGC | 1 | βAWLAYPDSVPYGSGC | 71 ± 26 | 2,900 ± 900 (2) | +++ |
| | dime | 1 | \| | (8) | N = 1.7 ± 0.1 | |
| | (C-ter) | | βAWLAYPDSVPYGSGC | | | |
| 2 | CGA-WLA-YR | 2 | CGAWLAYPDSVPYR | 7.9 ± 1.9 | 104 ± 1 | +++ |
| | dimer | 2 | \| | (4) | N = 2.0 ± 0 (2) | |
| | (N-ter) | | CGAWLAYPDSVPYR | | | |
| 3 | CcamGA = | 8 | C_{cam}GAWLAYPDSVPYR | 1,400 ± 240 | nd | inactive |
| | WLA-YR | | | (4) | | |
| 4 | CcamGA = WLA- | 18 | C_{cam}GAWLAYPDSVPYRPK- | 27 ± 7 | nd | +++ |
| | YRPK-bio | | bio | (4) | | |
| 5 | CGA-WLA-YRPK | 4 | CGAWLAYPDSVPYRPK | 0.65 ± 0.28 | 380 ± 80 (2) | 0.78 ± 0.025 |
| | dimer | 4 | \| | (9) | N = 2.2 ± 0.1 | (30) |
| | (N-ter) | | CGAWLAYPDSVPYRPK | | | |
| 6 | CGA-WLA-YRPKam | 5 | CGAWLAYPDSVPYRPKam | 0.40 ± 0.10 | nd | 0.80 ± 0.01 |
| | dimer | 5 | \| | (5) | | (19) |
| | (N-ter) | | CGAWLAYPDSVPYRPKam | | | |
| 7 | BA-WLA-YRPKC | 3 | βAWLAYPDSVPYRPKC | 0.50 ± 0.06 | 380 ± 80 (2) | 0,39 ± 0.02 |
| | dimer | 3 | \| | (3) | N = 2.0 ± 0.1 | (19) |
| | (C-ter) | | βAWLAYPDSVPYRPKC | | | |
| 8 | Linear YRPKam | 25 | βAWLAYPDSVPYRPKG- | 0.40 ± 0.07 | nd | 0.61 ± 0.02 |
| | dimer | 19 | -GAWLAYPDSVPYRPKCam | (4) | | (19) |
| | (head-to-tail) | | | | | |
| 9 | Click | 20 | K_{N3}AWLAYPDSVPYRPKam | 0.54 ± 0.09 | nd | 0.71 ± 0.02 |
| | A-WLA-YRPKam | 21 | \| | (4) | | (11) |
| | dimer | | PraGAWLAYPDSVPYRPKam | | | |
| | (N-ter) | | | | | |
| 10 | Lys-linked BA- | 22 | βAWLAYPDSVPYRPKK | 0.77 ± 0.02 | nd | 0.37 ± 0.01 |
| | WLA-YRPKam | 22 | / | (3) | | (14) |
| | dimer(N-ter) | | βAWLAYPDSVPYRPKK | | | |
| | m-ephrinA1 | | | 21 ± 8 | nd | 0.74 ± 0.02 |
| | (monomer) | | | (3) | | (8) |
| | ephrinA1Fc | | | nd | nd | 1.00 ± 0.03 |
| | (dimer) | | | | | (14) |

| | | | | | | | Solubility |
|---|---|---|---|---|---|---|---|
| Identi- | | | | | | | in PBS/H2O/ |
| fier | EC₅₀ | IC₅₀ | | Purity | Calculated | Observed | DMSO |
| number | PY588(nM)⁴ | PAKT(nM)⁴ | B_{lig}^{4,5} | (%) | mass (Da) | mass (Da) | (mg/ml) |
| 1 | in | in | in | 86 | 3,169.5 | 3,171.4 | <0.1/<0.1/5 |
| | progress | progress | progress | | | | |
| 2 | in | in | in | 95 | 3,193.6 | 3,192.9 | <0.1/<0.1/10 |
| | progress | progress | progress | | | | |
| 3 | nd | nd | nd | 95 | 1,654.9 | 1,654.8 | 5/5/10 |
| 4 | in | in | in | 97 | 2,106.5 | 2,106.2 | <0.1/5/10 |
| | progress | progress | progress | | | | |
| 5 | 0.53 ± 0.07 | 0.068 ± 0.011 | 0.81 ± 0.09 | 97 | 3,644.2 | 3,644.0 | 5/5/15 |
| | (17) | (17) | | | | | |
| 6 | 0.12 ± 0.01 | 0.043 ± 0.007 | 0.36 ± 0.09 | 99 | 3,642.2 | 3,641.0 | <0.1/5/10 |
| | (15) | (15) | | | | | |
| 7 | 2.6 ± 0.04 | 0.22 ± 0.04 | 1.28 ± 0.11 | 96 | 3,530.1 | 3,530.0 | 5/5/10 |
| | (13) | (13) | | | | | |
| 8 | 0.49 ± 0.06 | 0.12 ± 0.03 | 0.62 ± 0.13 | 95 | 3420.9 | 3421.0 | 1/5/10 |
| | (10) | (12) | | | | | |
| 9 | 0.22 ± 0.04 | 0.024 ± 0.004 | 0.93 ± 0.11 | 95 | 3,629.6 | 3,629.0 | <0.1/5/10 |
| | (7) | (12) | | | | | |
| 10 | 2.4 ± 0.2 | 0.63 ± 0.14 | 0.79 ± 0.11 | 96 | 3435.9 | 3435.5 | |
| | (10) | (14) | | | | | |
| | 28 ± 3 | 13 ± 2 | 0.30 ± 0.09 | | | | |
| | (6) | (6) | | | | | |
| | 0.81 ± v0.11 | 0.62 ± 0.10 | 0.00 ± 0.09 | | | | |
| | (7) | (9) | | | | | |

Although ITC measurements did not detect binding of free biotin to the EphA2 LBD, even when using high biotin concentration (1 mM; not shown), the crystal structures analyses described herein show that weak binding of the biotin moieties of two peptides to two EphA2 molecules anchored on the cell surface would be sufficient to promote receptor dimerization.

Further supporting the bivalent binding of the peptide agonists to two EphA2 molecules is the observation that the negative charge of the βA-WLA-YRPK C-terminus interacts with a neighboring EphA2 molecule in the crystal structure. It was found that this negative charge is required for EphA2 activation in cells in the absence of the C-terminal biotin as well as potentiates the effects of the biotin on EphA2 activation.

Further evidence shows that the localization of the biotin near the peptide C-terminus is critical, since an N-terminal biotin does not confer agonistic properties. The bivalent binding involving biotin is a distinctive feature of peptides targeting EphA2 because the three main EphA2 residues mediating biotin binding (Leu44, Thr45 and Tyr48), or homologous residues, are not all present in any other Eph receptor. In addition, biotinylated peptides binding to the ephrin-binding pocket of other Eph receptors do not function as agonists.

The bivalent binding mode described herein for the peptide agonists described herein is analogous to that observed for the dimeric forms of the ephrin-A ligands. Although the ephrin-A ligands are typically anchored on the cell surface through a glycosylphosphatidylinositol linkage, they can be released by metalloproteases as soluble proteins that also activate EphA2 signaling.

Dual-Dimerization Mechanism of EphA2 Allows for Techniques to Convert Described Peptides from Agonist to Antagonist

Interestingly, FRET measurements show that EphA2 can form some dimers in cells even in the absence of a bound ligand, for example when it is highly expressed in transiently transfected HEK293 cells. Furthermore, FRET analysis of the EphA2 L223R/L254R/V255R clustering interface mutant implicated this interface in the assembly of the EphA2 unliganded dimers. Destabilization of the clustering interface slightly decreases EphA2 oligomerization induced by YSA-GSGSK-bio, but to a much lesser extent than the G131Y mutation. This result indicated that the binding of peptide agonists such as YSA-GSGSK-bio induces dimerization of EphA2 monomers through the dimerization interface but also some assembly of larger EphA2 oligomers derived from pre-existing unliganded dimers and that these oligomers would use both interfaces. In contrast, dimers induced by monomeric ephrin-A1 are not affected by the EphA2 clustering interface triple mutation, demonstrating that the binding of monomeric ephrin-A1 disrupts the unliganded dimers whereas the binding of the peptides does not.

While the monovalent peptides can induce weak EphA2 tyrosine phosphorylation when present at very high concentrations, or when the receptor is highly expressed by transient transfection, at lower concentrations these peptides mainly function as antagonists that inhibit EphA2 signaling by an activating ligands such as ephrin-A1 Fc. Surprisingly, FRET studies have revealed that the non-biotinylated YSA-GSGSK increases the proportion of EphA2 dimers assembled through the clustering interface.

Others have reported a series of monomeric peptide derivatives obtained through replacement of various YSA residues with unnatural amino acids or chemical moieties (Gambini et al., 2018). These YSA derivatives were presumed to be agonists, although they are not biotinylated and lack a C-terminal negative charge. However, the mechanisms described herein teach away from this conclusion. The mechanisms described herein demonstrate that the peptides described in Gambini et al. 2018 instead function as antagonists, to be used when it is desirable to inhibit rather than activate EphA2.

The data described herein also do not support the critical importance attributed to Arg11 in the 135E2 peptide (Gambini et al., 2018). It was found that the corresponding Arg12 in βA-WLA-YRPK-bio interacts with Asp53 rather than Glu40, and only in one of the four molecules in the two structures described herein, while it does not make contacts with EphA2 in the other structures. Supporting the notion that the Arg does not make an important contribution to the interaction of YSA derivatives with EphA2. Arg12, however, plays a useful role in improving peptide solubility.

As a starting point to the novel modifications to the peptides described herein, the crystal structure for the complex formed by the binding of EphA2 to a known peptide (YSA) was characterized. In this initial characterization, a modified version of the peptide including a C-terminal GSGSK linker (SEQ ID NO: 14) with a biotin tag attached to the side chain of the lysine was used. The crystal structure of this peptide in complex with the EphA2 LBD at a resolution of 1.9 Å is described, for the first time, herein. The structure contains two peptide-EphA2 complexes in the asymmetric unit and in both complexes the electron density is well defined for the first 10 amino acids of the peptide, indicating that this part of YSA is mainly responsible for interaction with EphA2. The peptide binds to the ephrin-binding pocket of EphA2, which is the region that also interacts with the G-H loop of ephrin-A1. The first 4 amino acids of YSA bound to EphA2 closely overlap with residues F111 to F114 in the G-H loop of ephrin-A1 bound to the EphA2 LBD. In fact, the first 4 amino acids of YSA (YSAY (SEQ ID NO: 15)) conform to a WXXW motif (where W is an aromatic residue and X can be any residue) that is also present in the SWL peptide and the G-H loop of all the ephrin-A ligands. The remaining amino acids of YSA, however, are positioned differently from the corresponding residues of ephrin-A1. Pro5 introduces a kink in the peptide that is stabilized by a hydrogen bond with Ser7, so that the next residues occupy a groove of the EphA2 LBD that is only marginally involved in ephrin binding.

The YSA peptide forms an extensive network of hydrophobic and polar interactions with EphA2. Key interactions involve peptide Tyr1 (which binds to a hydrophobic pocket in EphA2 formed by Val72, Met73, Phe108, Pro109, and the Cys70-Cys188 disulfide bond) and Tyr4 (which is deeply buried in a hydrophobic pocket formed by Ile64, Met66, Thr101, Val161, Ala190, and Leu192). These interactions of the peptide are similar to those observed for Phe111 and Phe114 of ephrin-A1. Additional hydrophobic interactions are formed by peptide Pro5 with EphA2 Phe156 and Val161, peptide Pro9 with EphA2 Met55 and peptide Met10 with EphA2 Leu54 and Tyr65. Key polar interactions include a salt-bridge between peptide Asp6 and EphA2 Arg159 as well as hydrogen bonds between the backbone of peptide Ser2 and the side-chain of EphA2 Arg103, the backbone of peptide Pro5 and EphA2 Asn57, the backbone of peptide Val8 and the backbone of EphA2 Gln56, and the backbone of peptide Met10 with the backbone of EphA2 Leu54. Peptides built with Met11 and Ser12 and the GSGSK linker (SEQ ID NO: 14) in the structure were not developed because of their weak or absent electron density.

The most potent peptides described herein also have good solubility in aqueous solutions. The peptides described herein have the highest binding affinity among the EphA2-targeting peptides reported to date, by a surprisingly, significant amount. In addition, dimerization and immobilization on the surface of nanoparticles can further increase EphA2 targeting potency through avidity effects, as well as confer or potentiate agonistic properties. Disclosed herein are methods of dimerization and immobilization on the surface of nanoparticles to increase EphA2 targeting potency through avidity effects. The peptides described herein represent a valuable resource to modulate EphA2 by enabling potent and selective modification of the function of this receptor to increase or decrease signaling and to prevent binding of infectious agents.

The peptides described above are generally used to reduce inflammation. The peptides exert anti-inflammatory and also, immune-modulating effects. The peptides described herein can also be used to treat, prevent, or improve the symptoms of several other pathologies like cancer, auto-immune tissue destruction, and hyperglycemia.

The term “derivative” as used herein refers to peptides which have been chemically modified, including, but not limited to, by techniques such as biotinylation, ubiquitination, labeling, pegylation, glycosylation, or addition of other molecules. A molecule is also a “derivative” of another molecule when it contains additional chemical moieties not normally a part of the molecule. Such moieties can improve the molecule's solubility, absorption, biological half-life, etc. The moieties can alternatively decrease the toxicity of the molecule, eliminate or attenuate any undesirable side effect of the molecule, etc. Additional chemical moieties not normally a part of the molecule can increase the potency or binding affinity of said molecule.

Thus, in some embodiments, the peptides and methods disclosed herein comprise peptide derivatives, such as biotinylated peptides.

### Formulations of Therapeutically Effective Compositions of Peptides Described Herein

The administration of one or more peptides as disclosed herein or a mutant, variant, analog or derivative thereof may be by any suitable means that results in a concentration of the protein that treats the disorder. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously or intramuscularly), intraperitoneal, rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York, incorporated, herein, by reference in its entirety).

Pharmaceutical compositions according to the methods and compositions described herein may be formulated to release the active compound immediately upon administration or at any predetermined time or time period after administration. The latter types of compositions are generally known as controlled release formulations, which include (i) formulations that create substantially constant concentrations of the agent(s) of the compositions described herein within the body over an extended period of time; (ii) formulations that after a predetermined lag time create substantially constant concentrations of the agent(s) of the compositions described herein within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the therapeutic to a particular target cell type. Administration of the protein in the form of a controlled release formulation is especially preferred for compounds having a narrow absorption window in the gastrointestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the protein is formulated with appropriate excipients into a pharmaceutical composition that, upon administration, releases the protein in a controlled manner. Examples include single or multiple unit tablet or capsule compositions, oil solutions, suspensions, emulsions, microcapsules, molecular complexes, microspheres, nanoparticles, patches, and liposomes.

As used herein, the phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrastemal injection and infusion. The phrases “systemic administration,” “administered systemically”, “peripheral administration” and “administered peripherally” as used herein mean the administration therapeutic compositions other than directly into a tumor such that it enters the subject's system and, thus, is subject to metabolism and other like processes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent or encapsulating material, involved in maintaining the activity of or carrying or transporting the subject agents from one organ, or portion of the body, to another organ, or portion of the body. In addition to being “pharmaceutically acceptable” as that term is defined herein, each carrier must also be “acceptable” in the sense of being compatible with the other ingredients of the formulation. The pharmaceutical formulation comprising the one or more peptides as disclosed herein or a mutant, variant, analog or derivative thereof in combination with one or more pharmaceutically acceptable ingredients. The carrier can be in the form of a solid, semi-solid or liquid diluent, cream or a capsule. These pharmaceutical preparations are a further object of the methods and compositions described herein. Usually the amount of active compounds is between 0.1-95% by weight of the preparation, preferably between 0.2-20% by weight in preparations for parenteral use and preferably between 1 and 50% by weight in preparations for oral administration. For the clinical use of the methods described herein, targeted delivery compositions are formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the methods and compositions described herein in combination with one or more pharmaceutically acceptable ingredients. The carrier can be in the form of a solid, semi-solid or liquid diluent, cream or a capsule.

The term “pharmaceutically acceptable carriers” is intended to include all solvents, diluents, or other liquid vehicle, dispersion or suspension aids, surface active agents, isotonic agents, thickening or emulsifying agents, preservatives, solid binders, lubricants and the like, as suited to the particular dosage form desired. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its functional derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, com oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

### Parenteral Compositions

The pharmaceutical composition may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation or it may be presented as a dry powder to be reconstituted with water or another suitable vehicle before use. Apart from the active agent(s), the composition may include suitable parenterally acceptable carriers and/or excipients. The active agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the methods and compositions described herein may be in a form suitable for sterile injection. To prepare such a composition, the suitable active agent(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfate, sodium sulfite and the like; oil-soluble anti-oxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present compositions described herein include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect.

Methods of preparing these formulations or compositions include the step of bringing into association a compound of the compositions described herein with the carrier and, optionally, one or more accessory ingredients. In general, the formulations are prepared by uniformly and intimately bringing into association a compound of the compositions described herein with liquid carriers, or finely divided solid carriers, or both, and then, if necessary, shaping the product.

Formulations of the compositions described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present compositions described herein as an active ingredient. A compound of the present compositions described herein may also be administered as a bolus, electuary or paste.

Pharmaceutical compositions of the compositions described herein are suitable for parenteral administration comprise one or more peptides as disclosed herein or a mutant, variant, analog or derivative thereof in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions comprising one or more peptides as disclosed herein or a mutant, variant, analog or derivative thereof include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of the action of microorganisms may be ensured by the inclusion of various antibacterial and antifungal agents, including, but not limited to, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a par-enterally-administered drug form is accomplished by dissolving or suspending the drug in an oil vehicle.

Injectable forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Injectable formulations are also prepared by entrapping the drug, such as one or more peptides as disclosed herein or a mutant, variant, analog or derivative thereof in liposomes or microemulsions which are compatible with body tissue.

Regardless of the route of administration selected, the compounds of the present compositions described herein, which may be used in a suitable hydrated form, and/or the pharmaceutical compositions of the present compositions described herein, are formulated into pharmaceutically acceptable dosage forms by conventional methods known to those of ordinary skill in the art.

### Controlled Release Parenteral Compositions

Controlled release parenteral compositions may be in form of aqueous suspensions, microspheres, microcapsules, magnetic microspheres, oil solutions, oil suspensions, or emulsions. The composition may also be incorporated in biocompatible carriers, liposomes, nanoparticles, implants, or infusion devices.

Materials for use in the preparation of micro-spheres and/or microcapsules are, e.g., biodegradable/bio-erodible polymers such as polygalactia poly-(isobutyl cya-noacrylate), poly(2-hydroxyethyl-L-glutamine), poly(lactic acid), polyglycolic acid, and mixtures thereof. Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(capro-lactone), poly(lactic acid), poly(glycolic acid) or poly(ortho esters)) or combinations thereof.

### Solid Dosage Forms for Oral Use

Formulations for oral use include tablets containing the active ingredient(s) in a mixture with non-toxic pharmaceutically acceptable excipients, and such formulations are known to the skilled artisan.

These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the protein in a predetermined pattern (e.g., in order to achieve a controlled release formulation) or it may be adapted not to release the agent(s) until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate, may be employed.

The solid tablet compositions may include a coating adapted to protect the composition from unwanted chemical changes, (e.g., chemical degradation prior to the release of the active substances). The coating may be applied on the solid dosage form in a similar manner as that described in Encyclopedia of Pharmaceutical Technology, supra.

The compositions described herein may be mixed together in the tablet, or may be partitioned. In one example, a first agent is contained on the inside of the tablet, and a second agent is on the outside, such that a substantial portion of the second agent is released prior to the release of the first agent.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate, or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus, or spray drying equipment.

In solid dosage forms of the compositions described herein for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present compositions described herein, such as dragees, capsules, pills and granules, may optionally be scored or prepared with coatings and shells, such as enteric coatings and other coatings well known in the art of pharmacy. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally be of a composition that releases the active ingredient(s) only, or preferentially, in a certain portion of the gastrointestinal tract, optionally, in a delayed manner. Examples of embed-ding compositions which can be used include polymeric substances and waxes. The active ingredient can also be in micro-encapsulated form, if appropriate, with one or more of the above-described excipients. In one aspect, a solution of resolvin and/or protectin or precursor or analog thereof can be administered as eye drops for ocular neovascularization or ear drops to treat otitis.

Liquid dosage forms for oral administration of the compounds of the compositions described herein include pharmaceutically acceptable emulsions, microemulsions, solutions, suspensions, syrups and elixirs.

In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of one or more peptides as disclosed herein or derivative thereof include, but are not limited to, powders, sprays, ointments, pastes, creams, lotions, gels, solutions, patches and inhalants. The active compound may be mixed under sterile conditions with a pharmaceutically acceptable carrier, and with any preservatives, buffers, or propellants, which may be required.

The ointments, pastes, creams and gels may contain, in addition to an active compound of the compositions described herein, excipients, including, but not limited to, animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of the compositions described herein, excipients such as lactose, talc, silicic acid, aluminum hydroxide, calcium silicates and polyamide powder, or combinations thereof. Sprays can additionally contain customary propellants, such as chlorofluorohydrocarbons and volatile unsubstituted hydrocarbons, such as butane and propane.

Transdermal patches have the added advantage of providing controlled delivery of the compounds (resolvins and/or protectins and/or precursors or analogues thereof) of the present compositions described herein to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel. In another aspect, biodegradable or absorbable polymers can provide extended, often localized, release of polypeptide agents. The potential benefits of an increased half-life or extended release for a therapeutic agent are clear. A potential benefit of localized release is the ability to achieve much higher localized dosages or concentrations, for greater lengths of time, relative to broader systemic administration, with the potential to also avoid possible undesirable side effects that may occur with systemic administration.

Bioabsorbable polymeric matrix suitable for delivery of the one or more peptides as disclosed herein or a mutant, variant, analog or derivative thereof can be selected from a variety of synthetic bioabsorbable polymers, which are described extensively in the literature. Such synthetic bioabsorbable, biocompatible polymers, which may release proteins over several weeks or months can include, for example, poly-a-hydroxy acids (e.g. polylactides, polyglycolides and their copolymers), polyanhydrides, polyorthoesters, segmented block copolymers of polyethylene glycol and polybutylene terephtalate (Polyactive™), tyrosine derivative polymers or poly(ester-amides). Suitable bioabsorbable polymers to be used in manufacturing of drug delivery materials and implants have been previously described. The particular bioabsorbable polymer that should be selected will depend upon the particular patient that is being treated.

### Dosages

With respect to the therapeutic methods described herein, it is not intended that the administration of the one or more peptides as disclosed herein, or a derivative thereof, and be limited to a particular mode of administration, dosage, or frequency of dosing; the present methods and compositions described herein contemplate all modes of administration, including intramuscular, intravenous, intraperitoneal, intra-vesicular, intraarticular, intralesional, subcutaneous, or any other route sufficient to provide a dose adequate to treat the inflammation-related disorder. The therapeutic may be administered to the patient in a single dose or in multiple doses. When multiple doses are administered, the doses may be separated from one another by, for example, one hour, three hours, six hours, eight hours, one day, two days, one week, two weeks, or one month. For example, the therapeutic may be administered for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compositions. For example, the dosage of the therapeutic can be increased if the lower dose does not provide sufficient therapeutic activity.

While the attending physician ultimately will decide the appropriate amount and dosage regimen, therapeutically effective amounts of the one or more peptides as disclosed herein or a mutant, variant, analog or derivative thereof may be provided at a dose of 0.0001, 0.001, 0.01 0.1, 1, 5, 10, 25, 50, 100, 500, or 1,000 mg/kg. Effective doses may be extrapolated from dose-response curves derived from in vitro or animal model test bioassays or systems.

Dosages for a particular patient or subject can be determined by one of ordinary skill in the art using conventional considerations. A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the one or more peptides as disclosed herein or a mutant, variant, analog or derivative thereof and the condition of the patient, as well as the body weight or surface area of the patient to be treated. The size of the dose is also determined by the existence, nature, and extent of any adverse side-effects that accompany the administration of a particular vector, formulation, or the like in a particular subject. Therapeutic compositions comprising one or more peptides as disclosed herein, or a derivative thereof, are optionally tested in one or more appropriate in vitro and/or in vivo animal models of disease, such as models of cancer or inflammation, to confirm efficacy, tissue metabolism, and to estimate dosages. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of one or more peptides as disclosed herein or a mutant, variant, analog or derivative thereof. Administration can be accomplished via single or divided doses.

In determining the effective amount of one or more peptides as disclosed herein or a mutant, variant, analog or derivative thereof to be administered in the treatment or prophylaxis of disease the physician evaluates circulating plasma levels, formulation toxicities, and progression of the disease.

The efficacy and toxicity of the compound can be determined by standard pharmaceutical procedures in cell cultures or experimental animals, e.g., ED50 (the dose is effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred.

These compounds may be administered to humans and other animals for therapy by any suitable route of administration that works for small peptides, including orally, nasally, as by, for example, a spray, rectally, intravaginally, parenterally, intracisternally and topically, as by powders, ointments or drops, including buccally and sub-lingually.

Actual dosage levels of the active ingredients in the pharmaceutical compositions of compositions described herein may be varied so as to obtain an amount of the active ingredient which is effective to achieve the desired therapeutic response for a particular subject, composition, and mode of administration, without being toxic to the subject.

The selected dosage level will depend upon a variety of factors including the activity of the particular compound of the present compositions described herein employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

### Gene Therapy

One or more peptides as disclosed herein or derivative thereof can be effectively used in treatment by gene therapy. The general principle is to introduce the polynucleotide into a target cell in a patient.

Entry into the cell is facilitated by suitable techniques known in the art such as providing the polynucleotide in the form of a suitable vector, or encapsulation of the polynucleotide in a liposome.

A desired mode of gene therapy is to provide the polynucleotide in such a way that it will replicate inside the cell, enhancing and prolonging the desired effect. Thus, the polynucleotide is operably linked to a suitable promoter, such as the natural promoter of the corresponding gene, a heterologous promoter that is intrinsically active in liver, neuronal, bone, muscle, skin, joint, or cartilage cells, or a heterologous promoter that can be induced by a suitable agent.

Expression vectors compatible with eukaryotic cells, preferably those compatible with vertebrate cells, can be used to produce recombinant constructs for the expression of one or more peptides as disclosed herein or a mutant, variant, analog or derivative thereof, including fusion proteins with one or more peptides as disclosed herein or a mutant, variant, analog or derivative thereof. Eukaryotic cell expression vectors are well known in the art and are available from several commercial sources. Typically, such vectors are provided containing convenient restriction sites for insertion of the desired DNA segment. These vectors can be viral vectors such as adenovirus, adeno-associated virus, pox virus such as an orthopox (vaccinia and attenuated vaccinia), avipox, lentivirus, murine maloney leukemia virus, etc. Alternatively, plasmid expression vectors can be used.

Viral vector systems which can be utilized in the present methods and compositions described herein include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and G) a helper-dependent or gutless adenovirus.

The vector may or may not be incorporated into the cells genome. The constructs may include viral sequences for transfection, if desired. Alternatively, the construct may be incorporated into vectors capable of episomal replication, e.g. EPV and EBV vectors.

By “operably linked” is meant that a nucleic acid molecule and one or more regulatory sequences (e.g., a promoter) are connected in such a way as to permit expression and/or secretion of the peptide of the nucleic acid molecule when the appropriate molecules (e.g., transcriptional activator proteins) are bound to the regulatory sequences. Stated another way, the term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. An operatively linked polynucleotide which is to be expressed typically includes an appropriate start signal (e.g., ATG) and maintains the correct reading frame to permit expression of the polynucleotide sequence under the control of the expression control sequence, and production of the desired polypeptide encoded by the polynucleotide sequence.

As used herein, the terms “promoter” or “promoter region” or “promoter element” have been defined herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be ds-acting or may be responsive to trans-acting factors. Promoters, depending upon the nature of the regulation may be constitutive or regulated.

The term “regulatory sequences” is used inter-changeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Included in the term “regulatory elements” are nucleic acid sequences such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances, the promoter sequence is recognized by the synthetic machinery of the cell, or introduced synthetic machinery, required for initiating transcription of a specific gene.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, including, but not limited to, mutation, methylation etc.

In some embodiments, it can be advantageous to direct expression of one or more peptides as disclosed herein or a mutant, variant, analog or derivative thereof in a tissue- or cell-specific manner.

A gene or nucleic acid sequence can be introduced into a target cell by any suitable method. For example, one or more peptides as disclosed herein, or a derivative thereof, constructs can be introduced into a cell by transfection (e.g., calcium phosphate or DEAE-dextran mediated transfection), lipofection, electroporation, microinjection (e.g., by direct injection of naked DNA), biolistics, infection with a viral vector containing a muscle related transgene, cell fusion, chromosome-mediated gene transfer, microcell-mediated gene transfer, nuclear transfer, and the like. A nucleic acid encoding one or more peptides as disclosed herein, or a derivative thereof, can be introduced into cells by electroporation.

In certain embodiments, a gene or nucleic acid sequence encoding one or more peptides as disclosed herein or a mutant, variant, analog or derivative thereof can be introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioc-tadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PE1), and the like.

Methods known in the art for the therapeutic delivery of agents such as proteins and/or nucleic acids can be used for the delivery of a peptide or nucleic acid encoding one or more peptides as disclosed herein or a derivative thereof, e.g., cellular transfection, gene therapy, direct administration with a delivery vehicle or pharmaceutically acceptable carrier, indirect delivery by providing recombinant cells comprising a nucleic acid encoding a targeting fusion polypeptide of the compositions described herein.

Various delivery systems are known and can be used to directly administer therapeutic peptides as disclosed herein, or a derivative thereof, and/or a nucleic acid encoding one or more peptides as disclosed herein, or derivative thereof, e.g., encapsulation in liposomes, microparticles, microcapsules, recombinant cells capable of expressing the compound, and receptor-mediated endocytosis (see, e.g., Wu and Wu, 1987, J. Bioi. Chern. 262:4429-4432). Methods of introduction can be enteral or parenteral and include but are not limited to intradermal, intramuscular, intraperitoneal, intravenous, subcutaneous, pulmonary, intranasal, intraocular, epidural, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the compositions described herein locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

In another embodiment, the active agent can be delivered in a vesicle, in particular a liposome. In yet another embodiment, the active agent can be delivered in a controlled release system. In one embodiment, a pump may be used. In another embodiment, polymeric materials can be used.

Thus, a wide variety of gene transfer/gene therapy vectors and constructs are known in the art. These vectors are readily adapted for use in the methods described herein. By the appropriate manipulation using recombinant DNA/molecular biology techniques to insert an operatively linked polypeptide encoding nucleic acid segment into the selected expression/delivery vector, many equivalent vectors for the practice of the methods described herein can be generated.

### OTHER EMBODIMENTS

From the foregoing description, it will be apparent that variations and modifications may be made to the methods and compositions described herein to adopt it to various usages and conditions. Such embodiments are also within the scope of the following claims.

The disclosure also contemplates an article of manufacture, which is a labeled container for providing the one or more peptides as disclosed herein, or a mutant, variant, analog or derivative thereof. An article of manufacture comprises packaging material and a pharmaceutical agent of the one or more peptides as disclosed herein, or a derivative thereof, contained within the packaging material.

The pharmaceutical agent in an article of manufacture is any of the compositions described herein suitable for providing the one or more peptides as disclosed herein or a mutant, variant, analog or derivative thereof and formulated into a pharmaceutically acceptable form as described herein according to the disclosed indications. Thus, the composition can comprise the one or more peptides as disclosed herein, or a derivative thereof, or a DNA molecule which is capable of expressing such a peptide.

The article of manufacture contains an amount of pharmaceutical agent sufficient for use in treating a condition indicated herein, either in unit or multiple dosages. The packaging material comprises a label which indicates the use of the pharmaceutical agent contained therein.

The label can further include instructions for use and related information as may be required for marketing. The packaging material can include container(s) for storage of the pharmaceutical agent.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain a pharmaceutical composition including the pharmaceutical agent.

In preferred embodiments, the packaging material includes a label that is a tangible expression describing the contents of the article of manufacture and the use of the pharmaceutical agent contained therein.

### EXAMPLES

The following examples are provided to better illustrate the claimed methods and compositions and are not to be interpreted as limiting the scope of the methods and compositions described herein. To the extent that specific materials are mentioned, it is merely for purposes of illustration and is not intended to limit the methods and compositions described herein. One skilled in the art may develop equivalent means or reactants without the exercise of inventive capacity and without departing from the scope of the methods and compositions described herein.

### Example 1: Making Peptide Derivatives

### Peptides

Peptide identity and purity are documented by mass spectrometry and high-performance liquid chromatography (HPLC). The peptide solubility values are determined. Concentrated peptide stocks are prepared in DMSO or H₂O and stored frozen at −80° C.

### EphA2 Ligand Binding Doman (“LBD”) Expression and Purification

EphA2 receptors are expressed and purified. The DNA sequence coding for the EphA2 LBD (residues 28-200) with an additional C-terminal Ala-6×His-tag sequence (SEQ ID NO: 16) is cloned into a modified version of a pETNKI-LIC vector that encodes a N-terminal MASQGPG sequence (SEQ ID NO: 17) in a pET29 vector backbone. The EphA2 LBD is expressed in E. coli Origami 2(DE3) (Novagen) grown in 2×YT medium (BD Difco) at 20° C. overnight and purified using Ni-NTA agarose (Qiagen) followed by size-exclusion chromatography on a Superdex 75 10/300 GL column (GE Healthcare) equilibrated in 100 mM NaCl, 10 mM HEPES pH 7.9. The EphA2 LBD is concentrated to 5-7 mg/ml, flash frozen in aliquots, and stored at −80° C.

### Crystallization and Structure Solution

EphA2 LBD (7 mg ml⁻¹) is mixed with a 2-fold molar excess of one of the peptides listed in Table 2 dissolved to 2.9 mM in water, and initial crystals are obtained with the Hampton Index HT screen. Crystals are optimized with the Hampton Additive Screen HT, and may result in changes in the ratio of protein to precipitate volume, and by two rounds of crush seeding. Final crystals for structure solution are obtained by mixing 2.8 μl protein solution with 1 μl reservoir solution (0.09 M BIS-TRIS pH 5.5, 22.5% w/v PEG 3,350, 3% w/v 6-aminohexanoic acid) and equilibration against 50 μl reservoir solution at 20° C. in sitting-drop MRC 48-well plates (Molecular Dimensions). Clusters of plate-shaped crystals appear overnight. Crystals are cryoprotected by step-wise transfer to reservoir solutions with 5-15% glycerol and cryo-cooled in a nitrogen stream at 100 K. Diffraction data are collected on a rotating anode X-ray generator (Rigaku FR-E) at 100 K and processed in XDS and with software from the CCP4 suite. Phases are obtained using molecular replacement in Phaser with chain A of PDB ID 3HEI (Himanen et al., 2009) as search model. Model building and refinement are respectively performed in Coot (Emsley et al., 2010) or Refmac (Murshudov et al., 2011) and Phenix (Adams et al., 2010). The final model was validated using MolProbity (Chen et al., 2010).

Crystals for the complexes formed between EphA2 and the other peptides from Table 2 are grown in the same or similar conditions, for example, with 0.09 M Sodium-Acetate pH 4.5, instead of Bis-Tris pH5.5. The protein-to-precipitant drop ratio is in the range of 1.8-2.6 μl protein to 1111 precipitant for these crystals. Despite these similarities, the different complexes crystallize in different space groups, each with complexes in the asymmetric unit.

Isothermal Titration calorimetry (ITC)

For ITC, all peptides are dissolved in DMSO and both the EphA2 LBD and the peptides are diluted to obtain a final buffer containing 9.5 mM HEPES, pH 7.9, 95 mM NaCl, and 5% DMSO. The experiments are carried out at 296 K (23° C.) using an ITC200 calorimeter (Microcal). Two-microliter aliquots of a peptide solution are injected into the cell containing 205 μL EphA2 LBD. 200-400 μM peptides are titrated into 20-40 μM EphA2 LBD. Experimental data are analyzed using the Origin software package (Microcal). The integrated values for the reaction heats are normalized to the amount of injected peptide after blank subtraction.

### Modifications Increasing the Potency of YSA Derivatives

In addition to determining the crystal structure, the electron density in the interface between the two EphA2 molecules is also assessed. It has been observed that biotin interacts mainly with residues Thr45 and Tyr48 of the other EphA2 molecule in the asymmetric unit. Consistent with a contribution of the biotin to EphA2 binding, ELISAs measuring inhibition of ephrin-A5-EphA2 interaction reveal that the biotinylated peptides in Table 2 are more potent than peptides that do not contain the biotin.

Previous studies have shown that replacement of Tyr1 and Ser2 of YSA with SWL residues Trp2 and Leu3, respectively, improved peptide potency by about ˜2 fold. Since an alanine scan showed a favorable effect of replacing Ser1 in SWL with Ala, the WLAam peptide was modified by adding an N-terminal βAla. This unnatural amino acid was an improvement to Ala, its addition to the peptide resulted in improved peptide resistance to proteolytic degradation by plasma aminopeptidases. This replacement further increased potency by about ˜2 fold. Previous studies also showed that replacement of Met10 with Tyr (the corresponding residue in SWL), improved potency by another about ˜2 fold.

Previous studies determined that the crystal structure of a monomeric βA-WLA-Yam peptide in complex with the EphA2 LBD confirmed additional interactions with EphA2 that accounted for the increased potency. For example, extended hydrophobic interactions of the monomeric βA-WLA-Yam peptide were mediated by Trp2 and Tyr11. Further, βAla1 did not significantly interact with EphA2, this suggested that the observed ˜2-fold increase in potency due to the addition of βAla1 was caused by the elimination of the N-terminal positive charge of the Trp residue.

Previous studies have shown that the addition of Arg12, the residue present at the corresponding position of SWL, improved peptide solubility in aqueous solutions. Since Arg12 could introduce sensitivity to proteolytic degradation of C-terminal peptide extensions, a proline was included at position 13 because arginine followed by a proline is resistant to cleavage by trypsin-like proteases. In the previous studies, a lysine was also included at position 13 to allow attachment of biotin or other tags. Remarkably, the studies showed that an addition of both Pro13 and Lys14 increased potency by ˜7 fold. The binding affinity of monomeric βA-WLA-YRPK for the EphA2 LBD measured by isothermal titration calorimetry (ITC) was ˜200 nM, which was a 50-fold improvement compared to monomeric YSA-GSGSK-bio. As expected, the corresponding biotinylated peptide also exhibited much higher potency in ELISAs and much higher binding affinity measured by ITC. Replacement of Arg12 with Ser, to eliminate possible residual cleavage by trypsin-like proteases, yielded a peptide with only slightly decreased potency but with the disadvantage of not being soluble in aqueous solutions.

The crystal structures of the monomeric βA-WLA-YRPK-bio peptide in complex with the EphA2 LBD, determined in previous studies, explained the increased potency of this peptide. In one of the four complexes observed in the two structures, Arg12 interacted with EphA2 residues Asp53 and Tyr48. Peptide Pro13 packed against peptide Tyr11 and helped fill the hydrophobic pocket lined by EphA2 Leu54. In addition, the structures suggest that C-terminal amidation of monomeric βA-WLA-YRPK could further improve potency by eliminating the C-terminal negative charge positioned near the negatively charged Glu40 of EphA2. The amidated, monomeric βA-WLA-YRPKam and monomeric βA-WLA-YRPKam-bio peptides showed a ˜2-fold higher potency than the peptides with an unmodified C-terminus.

Importantly, YSA derivatives with greatly increased potency, such as monomeric βA-WLA-YRPK-bio, retained high specificity for EphA2 because even at concentrations 100-fold higher than the IC₅₀ value for inhibition of ephrin-A5-EphA2 binding, they did not inhibit ephrin binding to any other Eph receptor.

Based on these previous studies, the synthesis procedures described above were used to generate the dimeric peptides of Table 2.

### Example 2: C-Terminal Biotin and Negative Charge Potentiated the Agonistic Properties of YSA Derivatives

The YSA-GSGSK-bio peptide has been previously shown to be an agonist that induces EphA2 tyrosine phosphorylation and downstream signaling.

The two most potent biotinylated peptides of Table 2 are also agonists that induce high levels of EphA2 phosphorylation comparable to YSA-GSGSK-bio. However, as expected given their much higher potency, these two peptides are active at nanomolar concentrations. The C-terminal biotin promotes the agonistic activity of YSA derivative peptides. All biotinylated peptides strongly activate EphA2 and the precise position of the biotin (relative to the peptide residues interacting with the ephrin-binding pocket) does not have a strong effect on EphA2 activation. This is confirmed with the crystal structures. Thus, peptides with 1 to 7 residues between Prol 0, which is conserved in all YSA derivatives of Table 2, and the Lys-biotin residue, can all efficiently activate EphA2. In contrast, the non-biotinylated peptides either do not detectably activate EphA2 or are very weak activators that induce barely detectable EphA2 Y588 phosphorylation only when they are present at high concentrations.

C-terminal amidation of βA-WLA-YRPK-bio increases its binding affinity and potency in ELISAs but decreases its agonistic potency in cells, suggesting that the negative charge of the unmodified peptide C-terminus may play a role in EphA2 activation. Non-amidated βA-WLA-YRPK has the ability to activate EphA2 in cells, even though the concentrations needed are about 10-fold higher than for the biotinylated peptide and the maximal Y588 phosphorylation induced by saturating peptide concentrations is about 40% lower. The C-terminally amidated version of the peptide loses the ability to activate EphA2, consistent with a role of the C-terminal negative charge for EphA2 activation even in the absence of biotin.

A version of βA-WLA-YRPK with acetylation of the Lys14 side chain is examined to determine whether losing the positive charge in the side chain of Lys14 may contribute to the agonistic properties of the biotinylated peptides. The acetylated peptide had only slightly increased agonistic ability compared to the peptide comprising βA-WLA-YRPK, this suggests that the Lys14 positive charge has only minor detrimental effects on EphA2 activation. This is consistent with a direct effect of the biotin in promoting EphA2 activation in cells.

The crystal structures of the peptides in complex with the EphA2 LBD provides insights into the mechanisms underlying the agonistic properties of the peptides. In structures for the biotinylated peptides, the biotin binds at the interface between two EphA2 LBD molecules and makes similar contact with EphA2 residues. This raises the possibility that, in cells, two biotinylated peptides bridge two EphA2 molecules, with each peptide binding to the ephrin-binding pocket of an EphA2 molecule and the “biotin-binding pocket” of another EphA2 molecule. In addition, the C-terminus of βA-WLA-YRPK forms a salt bridge with Arg137 of the other EphA2 molecule in the asymmetric unit. The bivalent binding of biotinylated peptides could thus promote dimerization and reciprocal phosphorylation of EphA2 molecules, and in βA-WLA-YRPK-bio, this dimerization is further enhanced by the C-terminal negative charge.

Interestingly, four structures with three biotinylated peptides show EphA2 dimers that interact through the dimerization interface, whereas in the structure with the non-biotinylated βA-WLA-Yam peptide, the EphA2 molecules in the asymmetric unit interact differently, through an interface that is incompatible with the orientation of the receptors on the cell surface. According to the model described herein, a YSA derivative with biotin near the N-terminus should not efficiently activate EphA2 because such peptide would not simultaneously interact with the ephrin-binding pocket and the biotin-binding site.

The effects of YSA derivative dimeric peptides on AKT S473 phosphorylation are also noted, since EphA2 activation induced by ephrin-A ligands is known to inhibit AKT phosphorylation and activation. This confirms that the peptide agonists promote not only EphA2 activation but also downstream signaling.

### Example 3: The Dimeric Peptide Agonists Promote EphA2 Oligomerization Through the “Dimerization” Interface

Using a quantitative FRET approach in live cells, it is shown that, in transiently transfected HEK293 cells, dimeric YSA-GSGSK promotes the formation of EphA2 dimers that assemble through an extracellular interface known as the “clustering” interface. Thus, the dimeric peptide enhances the weak EphA2 dimerization observed in the absence of a bound ligand, which also occurs through the clustering interface. In contrast, the monomeric soluble form of ephrin-A1 induces the formation of EphA2 dimers that assemble through another extracellular interface known as the “heterodimerization” or “dimerization” interface. To understand the effects of the dimeric YSA derivatives with agonistic properties on the assembly of EphA2 oligomers (dimers or higher order clusters), quantitative FRET experiments are performed with HEK293 cells expressing EphA2 tagged at the C-terminus with a donor (mTURQ) or acceptor (EYFP) fluorescent protein.

The FRET measurements reveal that the compounds in Table 2 substantially increased the oligomeric fraction of EphA2 wild-type (WT) on the cell surface. The compounds in Table 2 also promote substantial oligomerization of the EphA2 L223R/L254R/V255R triple mutant, which has impaired ability to assemble through the clustering interface. In contrast, the biotinylated peptides of Table 2 have no effect on/reduced oligomerization of the EphA2 G131Y mutant, which has impaired ability to assemble through the dimerization interface. Comparison of the oligomerization curves of EphA2 WT and the two mutants in the absence of the compounds in Table 2 shows that the L223R/L254R/V255R mutations impairs dimerization while in the presence of the peptides. The G131Y mutation strongly impair dimerization and the triple mutation had a much smaller effect, suggesting that the biotinylated peptides mainly induce EphA2 dimerization through the dimerization interface. This approach is validated with the crystal structures. Thus, the FRET and X-ray crystallography data show that the peptide agonists induce EphA2 activation and downstream signaling by promoting interaction of receptor molecules on the cell surface through the dimerization interface.

### Example 4: The Compounds in Table 2 Lacking Agonistic Properties Inhibit Ephrin-Induced EphA2 Activation and Signaling

A number of the compounds in Table 2 appear to be inactive in the assays measuring EphA2 activation in cells. However, these peptides inhibit ephrin binding to EphA2 in ELISAs, some with low nanomolar potency. To determine whether they could also inhibit EphA2 activation by ephrin-A ligands in cells, the effects of the most potent compounds in Table 2 are examined. This reveals that the peptides inhibit EphA2 Y588 phosphorylation induced by ephrin-A1 Fc and thus can serve as antagonists. They also prevent the inhibitory effects of EphA2 activation on AKT.

While preferred embodiments of the present methods and compositions described herein have been shown, it will be obvious to those skilled in the art that such embodiments are provided by way of example only. Numerous variations, changes, and substitutions will now occur to those skilled in the art without departing from the methods and compositions described herein. It should be understood that various alternatives to the embodiments of the methods and compositions described herein may be employed in practicing the methods and compositions described herein. It is intended that the following claims define the scope of the methods and compositions described herein and that methods and structures within the scope of these claims and their equivalents be covered thereby.

### Example 5: Engineering Potent and Selective Dimeric Peptides with Different Configurations

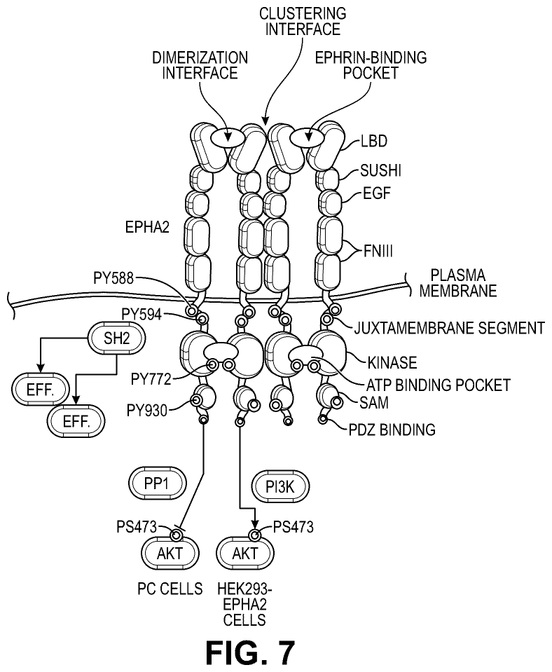

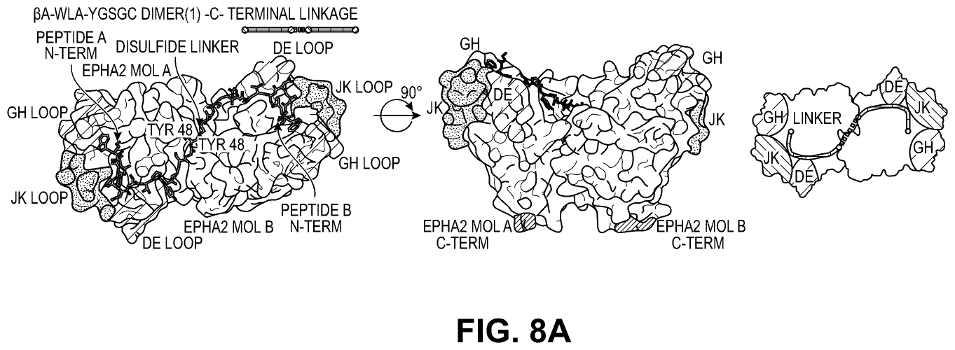

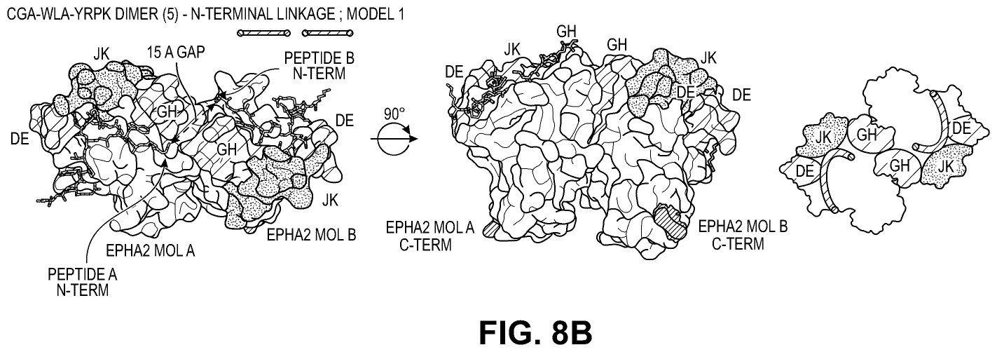

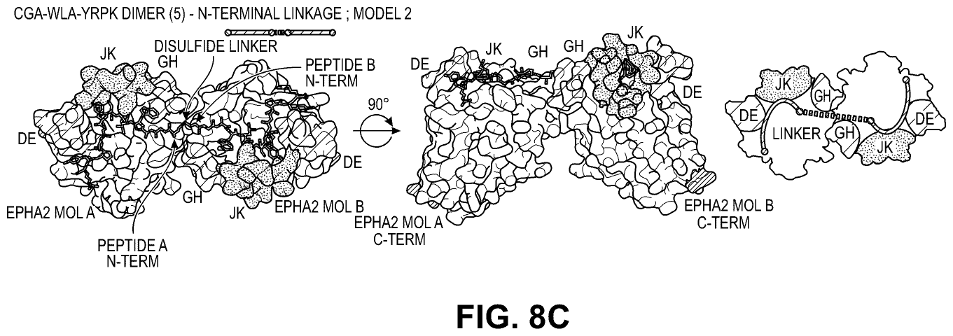

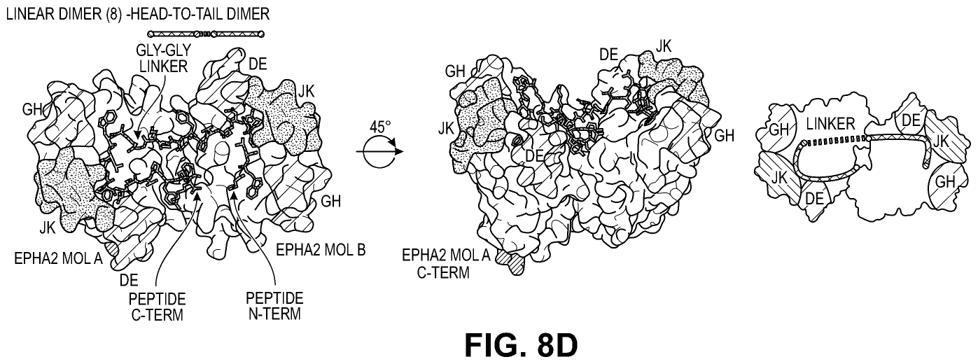

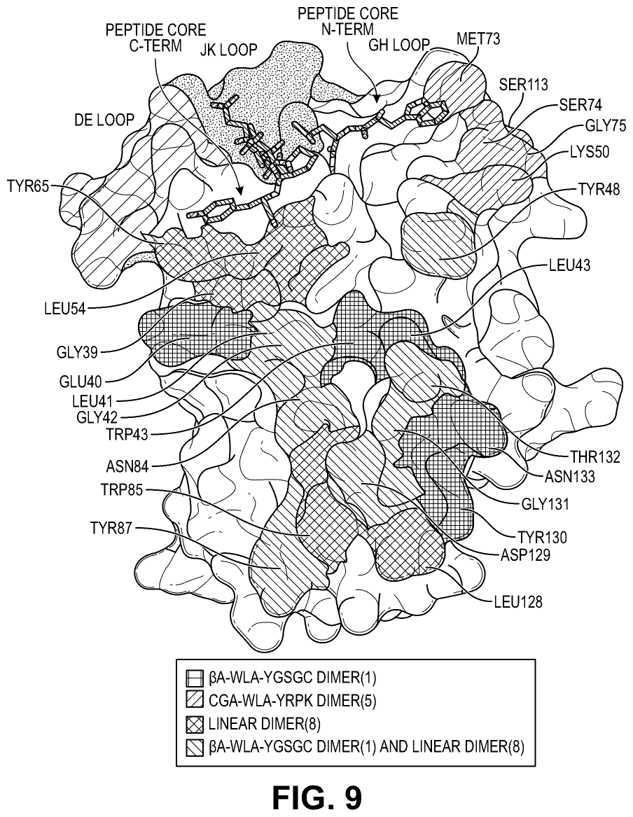

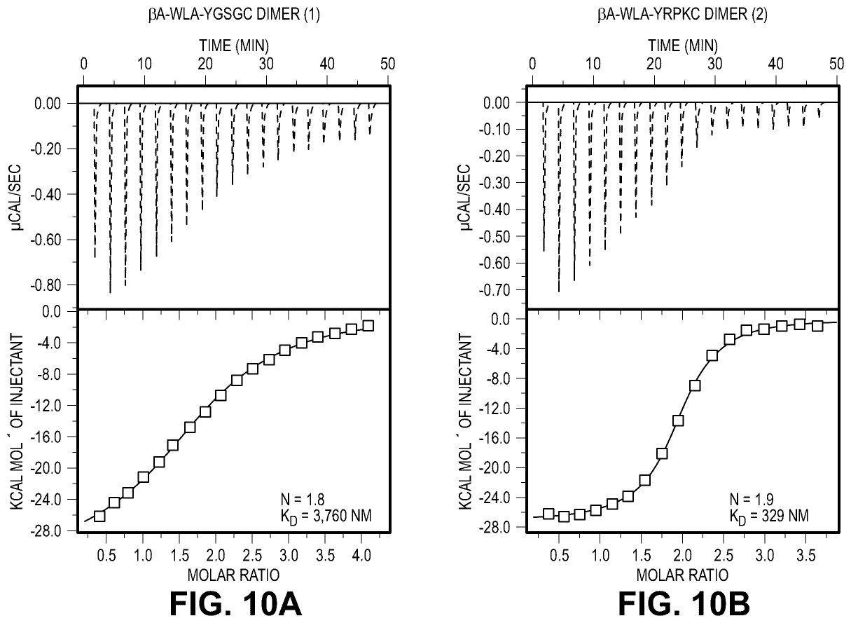

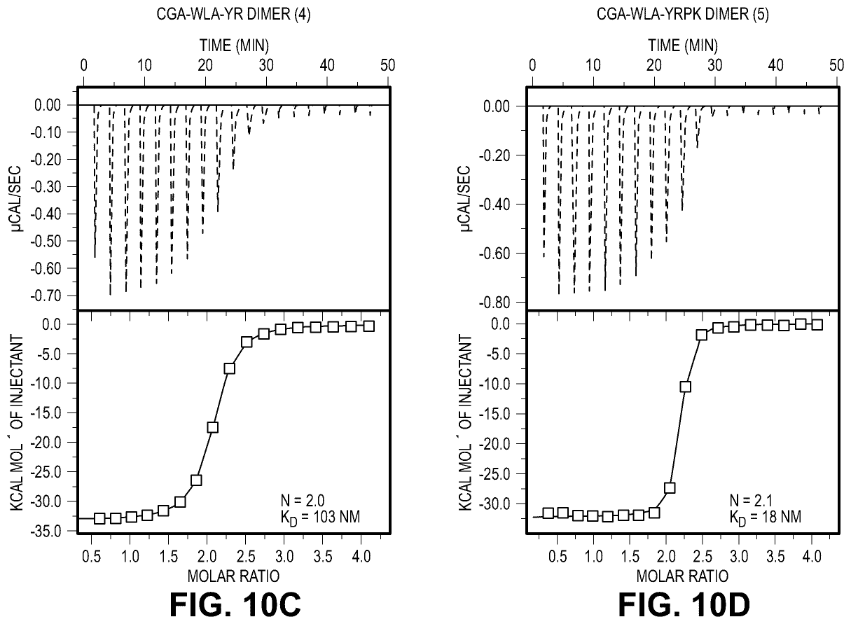

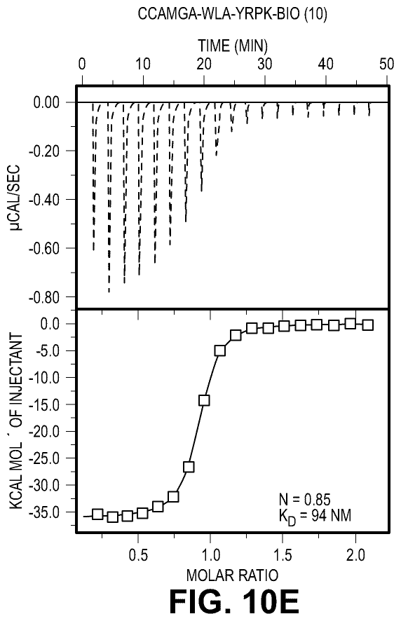

Ligands that promote EphA2 dimerization through the “dimerization” interface (FIG. 7), which was identified in crystal structures of the EphA2 extracellular region, were found to activate EphA2 kinase-dependent signaling. In silico modeling enabled us to design dimeric ligands predicted to induce EphA2 LBD dimerization through this interface by linking previously identified EphA2-targeting peptides through their C-termini (FIG. 8A). Dimer (1) was generated from the monomer (9*) and dimers (2) and (3) were generated from the monomer (19*), (Tables 3, 4 and 5). A C-terminal disulfide linkage was used in the case of dimers (1) and (2) and a more stable non-reducible linker in the case of dimer (3). In the in silico model, dimer (1) induces a symmetric EphA2 LBD dimer and occupies a channel formed in the dimerization interface by EphA2 residues Tyr48, Gly131 and Thr132 (FIGS. 8A and 9). The binding stoichiometry measured in isothermal titration calorimetry (ITC) experiments with the soluble EphA2 LBD confirmed that two EphA2 LBDs bind to each dimeric peptide (Tables 3 and 6; FIG. 10A,B).

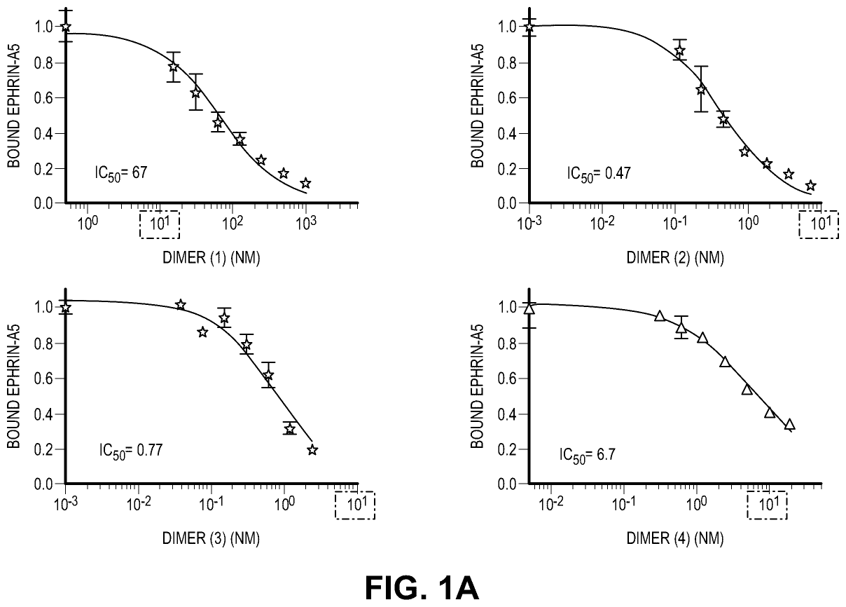

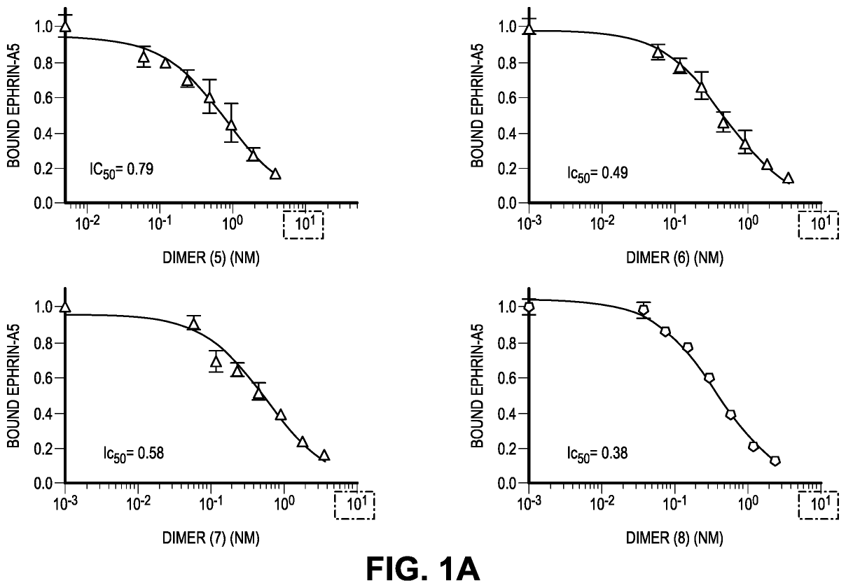

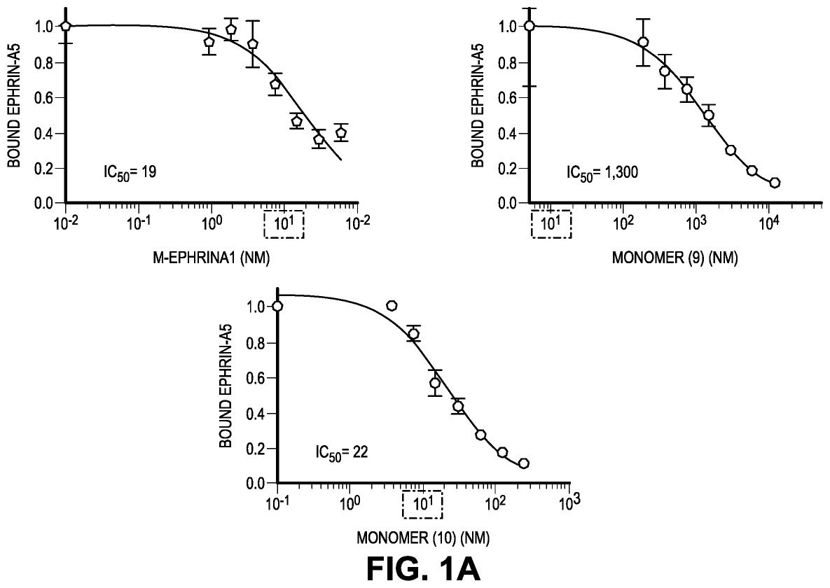

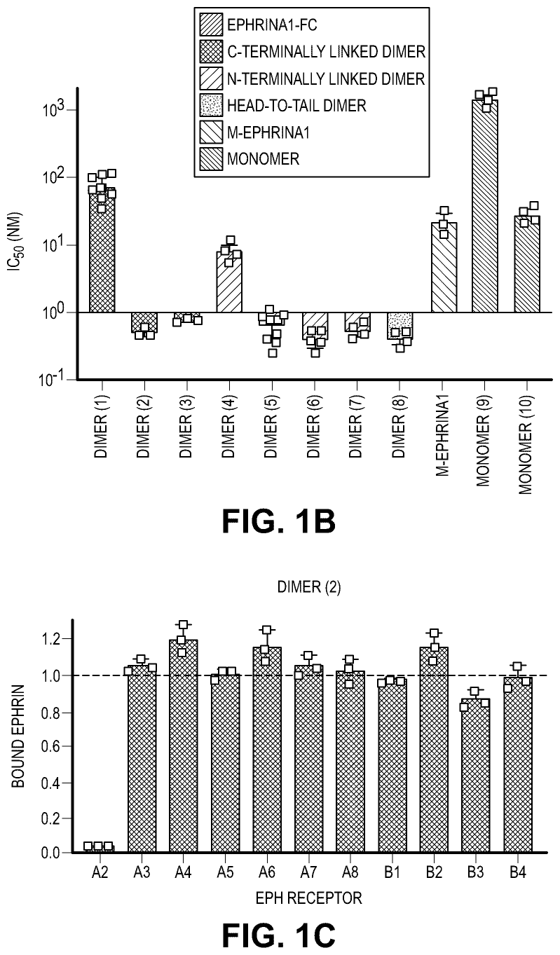

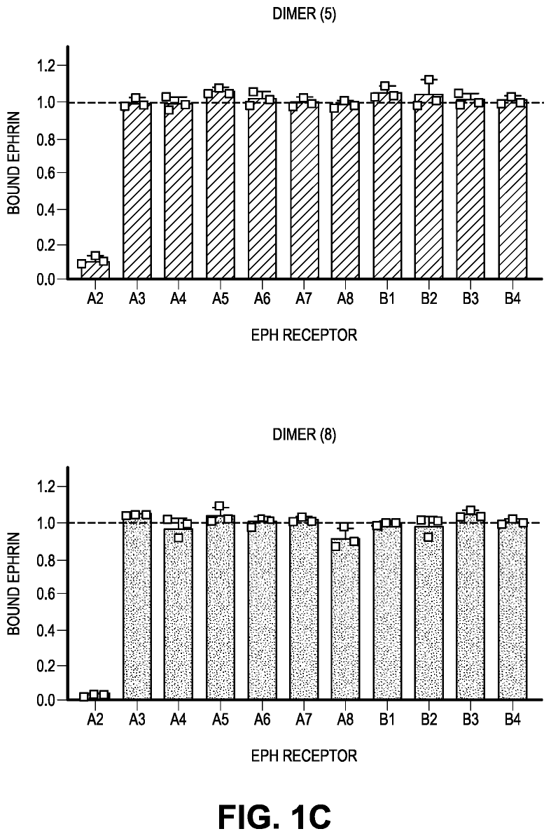

ELISAs measuring peptide-dependent inhibition of EphA2-ephrinA5 interaction revealed that the dimeric peptides are 6-40 times more potent than their monomeric precursors. The IC₅₀ values of 71 nM for dimer (1) compared to 410 nM for monomer (9*) and of 0.5 nM for dimer (2) and 0.77 nM for dimer (3) compared to 19 nM for monomer (19*) (FIG. 1A,B; Tables 3 and 5) are consistent with the expected increased binding avidity of dimeric ligands for EphA2 immobilized on the ELISA wells. On the other hand, the K_{d} values determined in ITC experiments dimers (1) and (2) reflect the affinity of the soluble monomeric EphA2 LBD for one of the two binding sites in the dimeric peptides, which should be largely independent of avidity effects (Table 3 and 6; FIG. 10A,B).

To obtain dimeric ligands with a completely different configuration, peptide monomers were joined through their N-termini. Dimer (4) from monomer (15*) was formed, dimer (5) from monomer (16*), and dimers (6) and (7) from monomer (19*) (Tables 3, 4 and 5). AN-terminal disulfide linkage was used in the case of dimers (4), (5) and (6) and a more stable non-reducible linkage in the case of dimer (7) (Tables 3 and 4).

Dimeric and Monomeric EphA2 Targeting Peptides
Dimeric peptides with different configurations and other EphA2 ligands

| | | | | | | | | | | |
|---|---|---|---|---|---|---|---|---|---|---|
| Identi- | | SEQ | | | | | | | | |
| fier | | ID | | ICsa (nM) | K_{D} (nM) | Etop | ECsa | Etop pAKT | ECsa pAKT | |
| number¹ | Name | NO | Sequence² | by ELISA³ | by ITC³ | pY588⁴ | pY588(nM)⁴ | inhlb⁴ | inhlb.(nM)⁴ | βu⁵ |

Dimers

| | | | | | | | | | | |
|---|---|---|---|---|---|---|---|---|---|---|
| 1 | βA-WLA-YGSGC | 11 | βAWLAYPDSVPYGSGC | 71 ± 26 | 2,900 ± 1,300(2) | 0.49 ± 0.06 | 150 ± 11 | 0.90 ± 0.04 | 39 ± 8 | 0.506 ± 0.12 |
| | dimer (C-ter) | | βAWLAYPDSVPYGSGC | (8) | N = 1.7 ± 0.2 | | (6) | | (6) | |
| 2 | βA-WLA-YRPKG | 3 | βAWLAYPDSVPYRPKC | 0.50 ± 0.06 | 380 ± 80 (2) | 0.50 ± 0.02 | 5.6 ± 0.6 | 0.86 ± 0.03 | 0.82 ± 0.15 | 0.73 ± 0.11 |
| | dimer (C-ter) | 3 | βAWLAYPDSVPYRPKC | (3) | N = 2.0 ± 0.2 | | (8) | | (7) | |
| 3 | Lys-linked | 22 | βAWLAYPDSVPYRPKK | 0.77 ± 0.02 | nd | 0.46 ± 0.02 | 2.6 ± 0.2 | 1.0 ± 0.03 | 0.42 ± 0.06 | 0.79 ± 0.09 |
| | dimer (C-ter) | 23 | βAWLAYPDSVPYRPK | (3) | | | (6) | | (8) | |
| 4 | GGA-WLA-YR | 2 | CGAWLAYPDSVPYR | 7.9 ± 1.9 | 104 ± 1 (2) | 0.94 ± 0.07 | 3.5 ± 0.2 | 0.90 ± 0.02 | 0.53 ± 0.08 | 0.47 ± 0.30 |
| | dimer (N-ter) | 2 | CGAWLAYPDSVPYR | (4) | N = 2.0 ± 0 | | (6) | | (8) | |
| 5 | CGA-WLA-YRPK | 4 | CGAWLAYPDSVPYRPK | 0.66 ± 0.28 | 21 ± 3 (2) | 0.87 ± 0.04 | 0.75 ± 0.07 | 1.0 ± 0.04 | 0.09 ± 0.02 | 066 ± 0.12 |
| | dimer (N-ter) | 4 | CGAWLAYPDSVPYRPK | (9) | N = 2.2 ± 0.2 | | (6) | | (6) | |
| 6 | CGA-WLA-YRPKaes | 5 | CGAWLAYPDSVPYRPKan | 0.40 ± 0.10 | nd | 0.98 ± 0.08 | 0.36 ± 0.06 | 0.97 ± 0.03 | 0.08 ± 0.02 | 0.51 ± 0.13 |
| | dimer (N-ter) | 5 | CGAWLAYPDSVPYRPKan | (5) | | | (6) | | (6) | |
| 7 | Click dimer | 21 | K_{N3}AWLAYPDSVPYRPKam | 0.54 ± 0.09 | nd | 0.98 ± 0.0 | 0.64 ± 0.06 | 0.87 ± 0.02 | 0.08 ± 0.01 | 0.50 ± 0.10 |
| | (N-ter) | 22 | PᵣₐGAWLAYPDSVPYRPKam | (4) | | | (8) | | (8) | |
| 8 | anaadimer | 23 | βAWLAYPDSVPYRPK | 0.40 ± 0.07 | nd | .76 ± 0.0 | 0.73 ± 0.06 | 0.87 ± 0.02 | 0.09 ± 0.01 | 0.62 ± 0.06 |
| | (head total) | 6 | GAWLAYPDSVPYRPKₐₘ | (4) | | | (6) | | (6) | |
| | ephrinA1-FC | | | nd | nd | .00 ± 0.0 | 3.8 ± 0.2 | 1.0 ± 0.01 | 1.7 ± 0.2 | 0.00 |
| | (dimer) | | | | | | (14) | | (14) | |

monomers

| | | | | | | | | | | |
|---|---|---|---|---|---|---|---|---|---|---|
| | m-ephrinA1 | | | 21 ± 8 | nd | .36 ± 0.03 | 74 ± 6 | 1.0 ± 0.02 | 27 ± 2 | 054 ± 0.08 |
| | | | | (3) | | <0. | (14) | | (14) | |
| 9 | CcamGA-WLA-YR | 8 | C_{cam}GAWLAYPDSVPYR | 1,400 ± 240 | nd | .48 ± 0.0 | nd | nd | nd | nd |
| | | | | (4) | | | | | | |
| 10 | CcamGA-WLA- | 18 | C_{cam}GAWLAYPDSVPYRPK- | 27 ± 7 | 80 ± 21(2) | .42 ± 0.02 | 180 ± 20 | 1.06 ± 0.03 | 42 ± 6 | 0.64 ± 0.09 |
| | YRPK-bio | | bio | (4) | N = 0.8 ± 0.1 | | (9) | | (9) | |
| 2* | YSA-GSGSK-bio | 24 | YSAYPDSVPMMSGSGSK- | 850 ± 440 | 9,800 ± 0(2) | 0.49 ± 0.06 | 3,900 ± 380 | 098 ± 0.02 | 600 ± 82 | 0.84 ± 0.09 |
| | | | bio | (43) | | | (8) | | (6) | |

TABLE 4
Purity and mass of EphA2-targeting peptides

| | | | Calculated | Observed | Solubility in |
|---|---|---|---|---|---|
| Identifier | | Purity | mass | mass | PBS/H20/DMSO |
| number | Name | (%) | (Da) | (Da) | (mg/ml) |
| 1 | βA-WLA-YGSGC dimer | 86 | 3,169.50 | 3,171.40 | <0.1/ |
| | (C-ter) | | | | <0.1/5 |
| 2 | βA-WLA-YRPKC dimer | 96 | 3,530.10 | 3,530.00 | 5/5/2010 |
| | (C-ter) | | | | |
| 3 | Lys-linked dimer | 96 | 3,435.90 | 3,435.50 | <0.1/5/ |
| | (C-ter) | | | | 10 |
| 4 | CGA-WLA-YR dimer | 95 | 3,193.60 | 3,192.90 | <0.1/ |
| | (N-ter) | | | | <0.1/10 |
| 5 | CGA-WLA-YRPK dimer | 97 | 3,644.20 | 3,644.00 | 5/5/2015 |
| | (N-ter) | | | | |
| 6 | CGA-WLA-YRPKam | 99 | 3,642.20 | 3,641.00 | <0.1/5/ |
| | dimer (N-ter) | | | | 10 |
| 7 | Click dimer | 95 | 3,629.60 | 3,629.00 | 1/5/2010 |
| | (N-ter) | | | | |
| 8 | linear dimer | 95 | 3,420.90 | 3,421.00 | 5/5/2010 |
| | (head-to-tail) | | | | |
| 9 | C_{cam}GA-WLA-YR | 95 | 1,654.90 | 1,654.80 | 5/5/2010 |
| 10 | C_{cam}GA-WLA-YRPK-bio | 97 | 2,106.50 | 2,106.20 | <0.1/5/ |
| | | | | | 10 |

TABLE 6
Thermodynamic parameters of peptide-EphA2 interaction

| Identi- | | | ΔG | ΔH | -TΔS | |
|---|---|---|---|---|---|---|
| fier | Name | K(nM)¹ | (kcal/mol) | (kcal/mol) | (kcal/mol) | N |
| 1 | βA-WLA- | 2,900 ± 1,300 | −7.55 ± 0.27 | −23.85 ± 11 | 16.30 ± 11 | 1.7 ± 0.2 |
| | YGSGC | (2) | | | | |
| | dimer | | | | | |
| 2 | βA-WLA- | 380 ± 80 | −8.71 ± 0.13 | −28.17 ± 1.42 | 19.46 ± 0.155 | 2.0 ± 0.2 |
| | YRPKC | (2) | | | | |
| | dimer | | | | | |
| 4 | CGA-WLA- | 104 ± 1 | −9.47 ± 0.01 | −33.12 ± 0.04 | 23.65 ± 0.02 | 2.0 ± 0.02 |
| | YR dimer | (2) | | | | |
| 5 | CGA-WLA- | 21 ± 3 | −10.41 ± 0.10 | −32.34 ± 0.21 | 21.93 ± 0.31 | 2.2 ± 0.2 |
| | YRPK | (2) | | | | |
| | dimer | | | | | |
| 10 | CGA-WLA- | 80 ± 21 | −9.63 ± 0.15 | −35.12 ± 1.3 | 25.49 ± 1.49 | 0.80 ± 0.07 |
| | YRPK- | (2) | | | | |
| | bio | | | | | |

ΔG is the change in Gibbs energy, ΔH is the change in enthalpy, T is the absolute temperature, ΔS is the change in entropy and N is the binding stoichiometry.
¹Averages ± SD are shown. The number of experiments is indicated in parentheses.

Dimers (5) and (6) have an amidated C-terminus to increase binding affinity and prevent the possible interaction of the C-terminal carboxylic acid with a neighboring EphA2 LBD.

ITC experiments confirmed the expected binding stoichiometry of two EphA2 LBDs binding to one N-terminally linked dimer (4) or (5) (Tables 3 and 6; FIG. 10C,D). In silico modeling of the two monomeric peptides used to generate dimer (5) in complex with dimeric EphA2 LBDs showed that the peptide N-termini are too far apart (˜15 Å) to form a disulfide bond (FIG. 8B). Manual adjustments of the model to bring the N-terminal cysteines in close proximity to each other required slight translation and tilting by −45° of each EphA2 LBD (FIG. 8C). In this orientation there are only minor contacts between the two EphA2 LBDs (including contacts involving Lys50, Gly75 and Ser113; FIG. 9). Inhibition of ephrin-A5 binding to immobilized EphA2 in ELISAs indicated that the N-terminally linked dimers are also 30-140 times more potent than the corresponding monomers, with IC₅₀ values of 7.9 nM for dimer (4) versus 390 nM for monomer (15*), 0.65 nM for dimer (5) versus 55 nM for monomer (16*), 0.40 nM for dimer (6) and 0.54 nM for dimer (7) versus 19 nM for monomer (19*) (FIG. 1A,B; Tables 3 and 5). The K_{d} values measured in ITC experiments with the soluble EphA2 LBD are 104 nM for dimer (4) and 21 nM for dimer (5) (Tables 3 and 6; FIG. 10C, 10D), supporting the notion that the subnanomolar potency of the dimers in ELISAs is due to avidity effects.

To evaluate monomeric peptides more similar in their first three residues to the dimers, new monomers (9) and (10) were also generated. These monomers contain a N-terminal carbamidomethylcysteine, which mimics the cysteine present in the dimers but cannot form a disulfide bond, followed by a glycine and an alanine instead of b-alanine (Tables 3 and 4). Monomers (9) and (10) are much less potent than the corresponding dimers (4) and (5) (FIGS. 1A, 1B and 10E; Table 3), consistent with the notion that the high potency of the N-terminally linked dimers is due to increased avidity and not to the N-terminal modifications.

To obtain a third dimeric peptide configuration, an asymmetric dimer was designed in which two monomer (19*) sequences are synthesized one after the other with an intervening GlyGly linker to yield a linear “head-to-tail” dimer (8) (Tables 3, 4 and 5). The second peptide sequence starts with alanine instead of β-alanine, since protection from digestion by aminopeptidases is not needed for an internal residue. The IC₅₀ value for dimer (8) in ELISAs is also subnanomolar (FIG. 1A, 1B; Table 3), again suggesting increased potency due to avidity effects. In silico modeling suggests that the two EphA2 LBDs in complex with dimer (8) form a symmetric dimer and utilize an interface that partially overlaps with that induced by the C-terminally linked dimer (1), including Tyr48, Gly131 and Thr132 (FIGS. 8D and 9). However, the interface is distinct from the dimerization interface induced by dimer (1) because one of the EphA2 LBDs bound to dimer (8) is rotated by about 70° and shifted by about 11 Å with respect to the hypothetical plane between the two EphA2 LBDs.

An important feature of the original YSA peptide and its monomeric derivatives is that they specifically bind to EphA2, whereas the ephrinA ligands promiscuously binds to all EphA receptors (9, 31, 37, 53). Despite their very high potency, dimeric peptides (2), (5) and (8)—representing the three different configurations—are also highly selective for EphA2 and do not bind to other Eph receptors (FIG. 1C). Thus, dimers (1) through (8) represent a collection of very potent and diverse dimeric ligands that are highly specific for EphA2, enabling us to study how differences in ligand configuration, potency and linker type affect EphA2 signaling responses.

### Example 6: Dimeric Peptides which Potently Activate EphA2 Regardless of their Dimeric Configuration

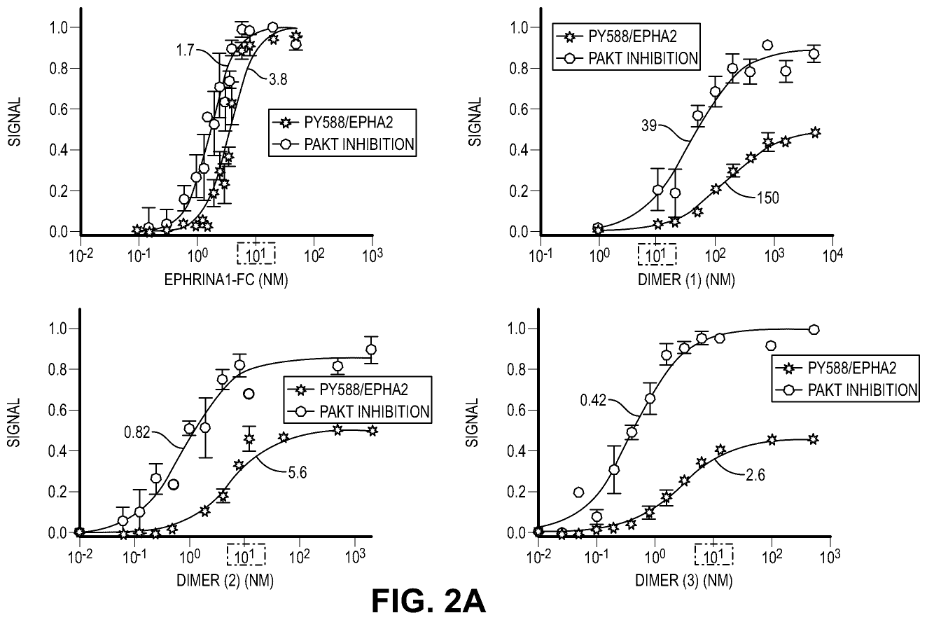

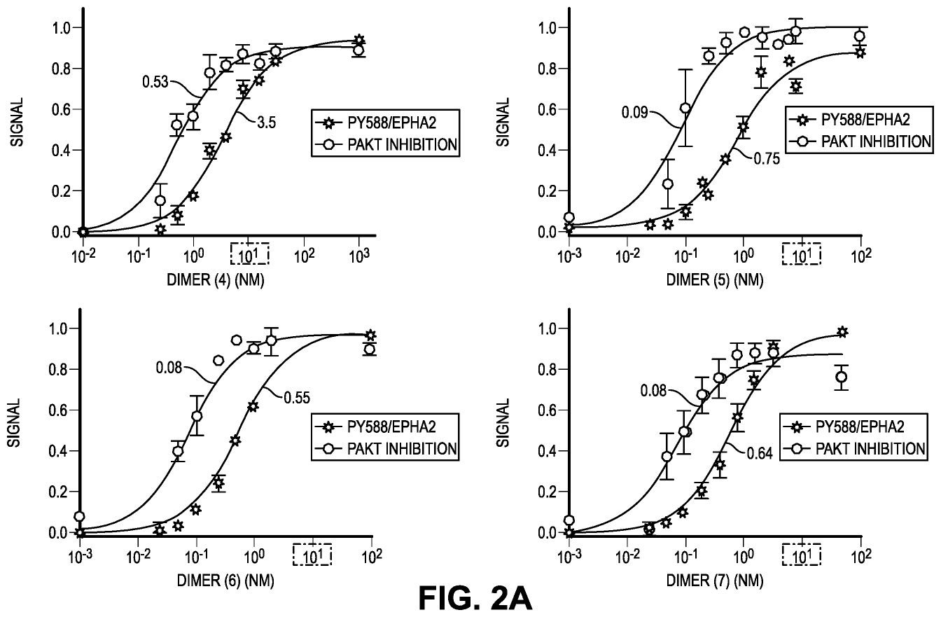

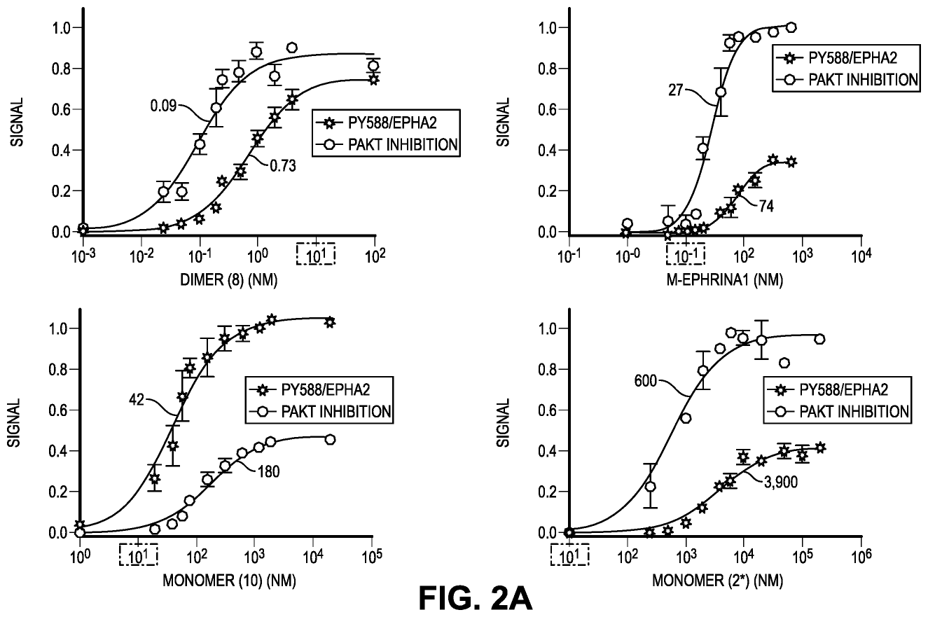

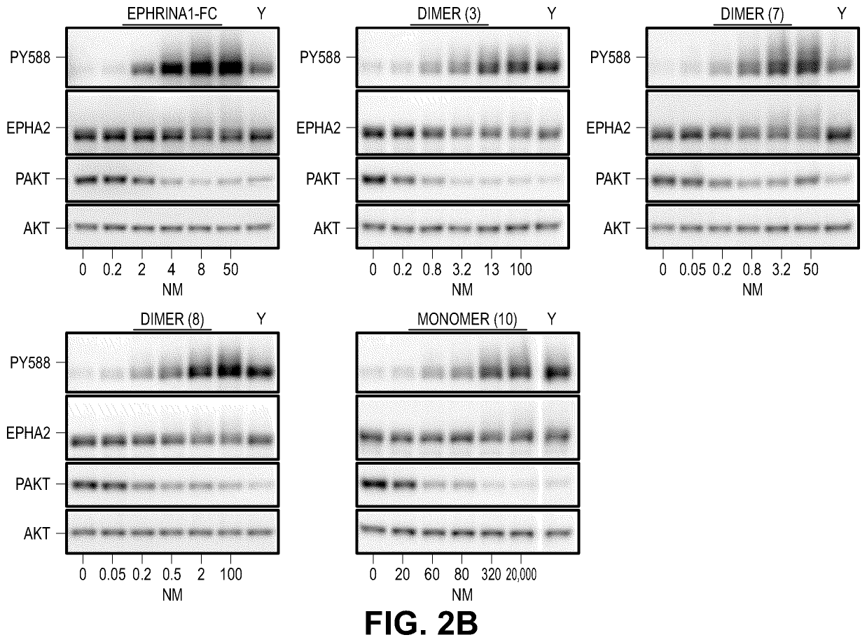

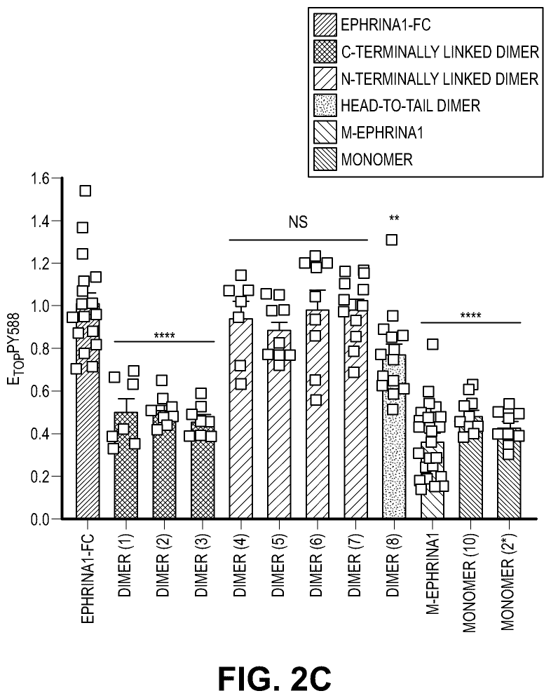

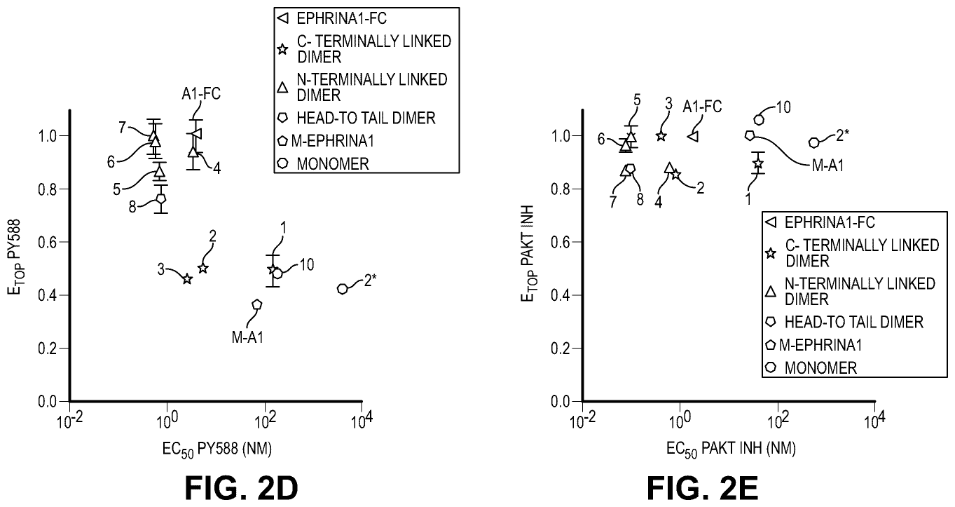

To examine the agonistic properties of the different dimeric peptide ligands, EphA2 autophosphorylation on tyrosine 588 (Y588) was measured, which is indicative of receptor activation and mediates binding of SH2 domain-containing proteins that link EphA2 to various downstream signaling pathways (FIG. 7). For these experiments, PC3 prostate cancer cells were stimulated with the dimeric peptides because EphA2 is the prevalent endogenously expressed EphA receptor in these cells, allowing comparisons with ephrins. Peptides with all three different dimeric configurations were found to readily induce robust EphA2 autophosphorylation (FIG. 2A-2D). The agonistic potency of the dimers varies according to the potency of their monomeric precursors, as expected, with dimers (1) and (4) exhibiting 10-60 times lower potency than the other dimers with similar configuration (FIG. 2D; Table 3). However, dimers are much more potent than monomers (FIG. 2A, 2B, 2D; Table 3), likely due to their increased binding avidity for EphA2 on the cell surface. The potency of the dimers also depends on their configuration; the N-terminally linked and head-to-tail dimers exhibit higher potency than the C-terminally linked dimers, with the best EC₅₀ values as low as 0.55 to 0.75 nM for dimers (5) through (8). Another difference that correlates with dimeric configuration is that the three C-terminally linked dimers have lower efficacy (i.e. they induce lower maximal EphA2 Y588 phosphorylation, Eₜₒₚ pY588; FIG. 2A, 2C, 2D; Table 3). In contrast, the nature of the linker used for dimerization does not seem to have major effects on potency.

The engineered ligand most widely used to activate EphA2 signaling is the dimeric ephrinA1-Fc, in which the ephrinA1 extracellular region is fused to the dimeric Fc portion of an antibody. Treatment of cells with ephrinA1-Fc is known to induce EphA2 oligomerization, autophosphorylation on tyrosine residues including Y588, and downstream signaling. Remarkably, ephrinA1-Fc (EC₅₀=3.8 nM) is substantially less potent than peptide dimers (5) through (8) (FIG. 2A,B,D; Table 3).

The monomeric m-ephrinA1 ligand, which is also known to induce EphA2 autophosphorylation, was as expected less potent than ephrinA1-Fc (FIG. 2A, 2D; Table 3). The new monomeric C_{cam}GA-WLA-YRPK-bio (10) peptide, but not C_{cam}GA-WLA-YR (9), also induced EphA2 autophosphorylation (FIG. 2A-D; Table 3), in agreement with our observation that a C-terminal biotin confers agonistic properties to this class of monomeric peptides. Thus, C_{cam}GA-WLA-YRPK-bio (10) represents a new monomeric EphA2 agonist with nanomolar potency.

EphA2 activation by ephrinA1-Fc is also known to strongly inhibit AKT in PC3 cells, which can be monitored by measuring the decrease in AKT phosphorylation on S473 (FIGS. 2A, 2B, 2E and 7). All peptide agonists and m-ephrinA1 were found also inhibit AKT in a concentration-dependent manner (FIG. 2A, 2B, 2E; Table 3). Thus, like the ephrins, all peptide agonists promote not only EphA2 autophosphorylation but also downstream signaling. However, a difference between the peptides and ephrinA1 is that dose-response curves with a Hill coefficient of 1 satisfactorily describe the data obtained with the peptides but not the data obtained with ephrinA1-Fc and m-ephrinA1, for which a Hill coefficient of 2 yields a much better fit. This suggests positive cooperativity in the binding of both monomeric and dimeric forms of ephrinA1 to EphA2.

### Example 7: Kinetics of EphA2 Signaling Differ Depending on the Activating Ligand

As mentioned above, EphA2 Y588 phosphorylation levels induced by stimulating PC3 cells for 15 min with saturating ligand concentrations (inducing maximal Eₜₒₚ pY588) are lower for the C-terminally linked dimers and the monomeric ligands than for the N-terminally linked dimers and head-to-tail dimer (8), which are similar to the reference ligand ephrinA1-Fc (FIG. 2C, 2D; Table 3), suggesting that the configuration of the dimers affects signaling features. For example, C-terminally linked dimers may be partial agonists that are able to achieve only low maximal EphA2 Y588 phosphorylation. Alternatively, different ligands may regulate EphA2 phosphorylation with distinct kinetics. If the EphA2 phosphorylation kinetics are slower for C-terminally linked dimers, peak phosphorylation levels may not be reached by 15 min. If the kinetics of dephosphorylation are faster, peak phosphorylation may have already declined by 15 min. To distinguish among these possibilities, and to further characterize the activities of the different ligands, time course experiments were performed with saturating concentrations of peptides representative of each group and ephrinA1-Fc.

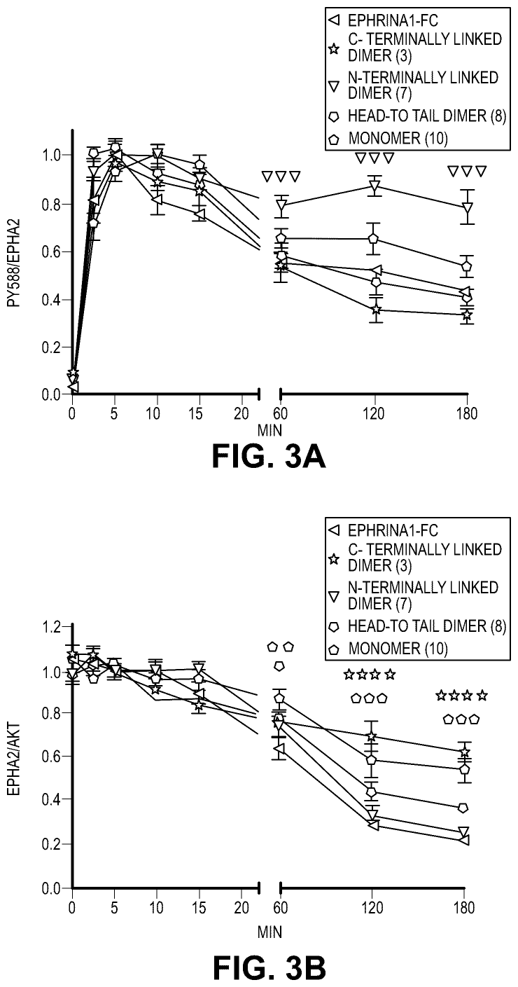

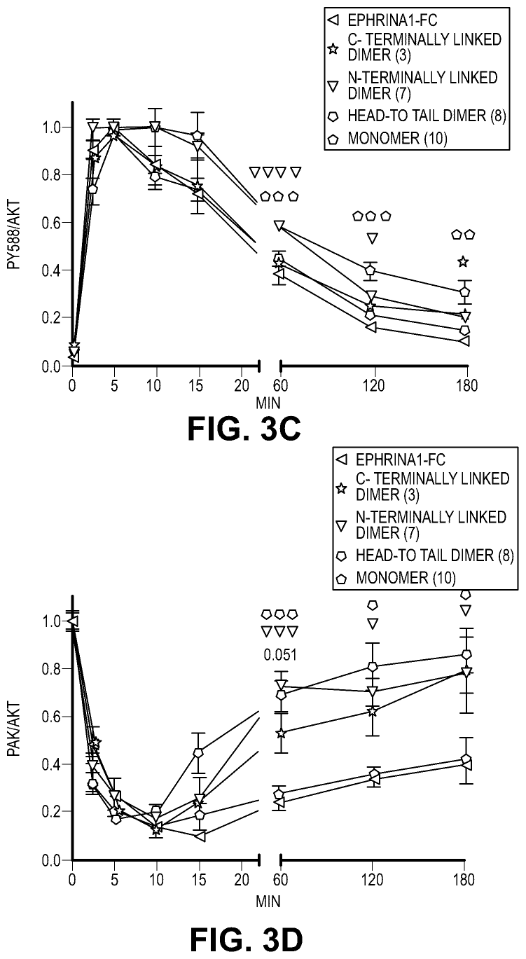

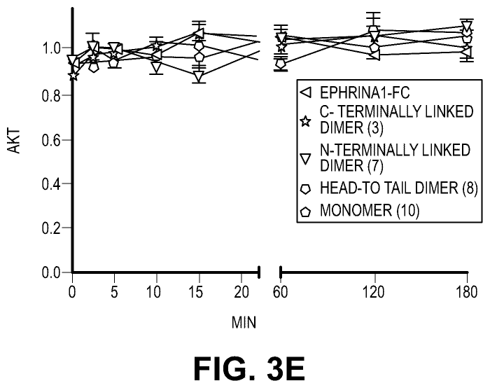

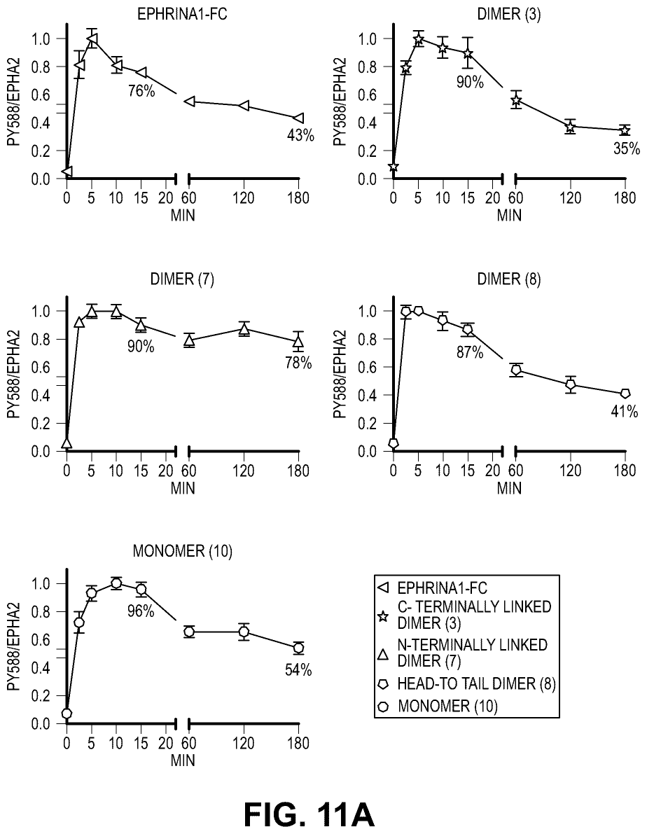

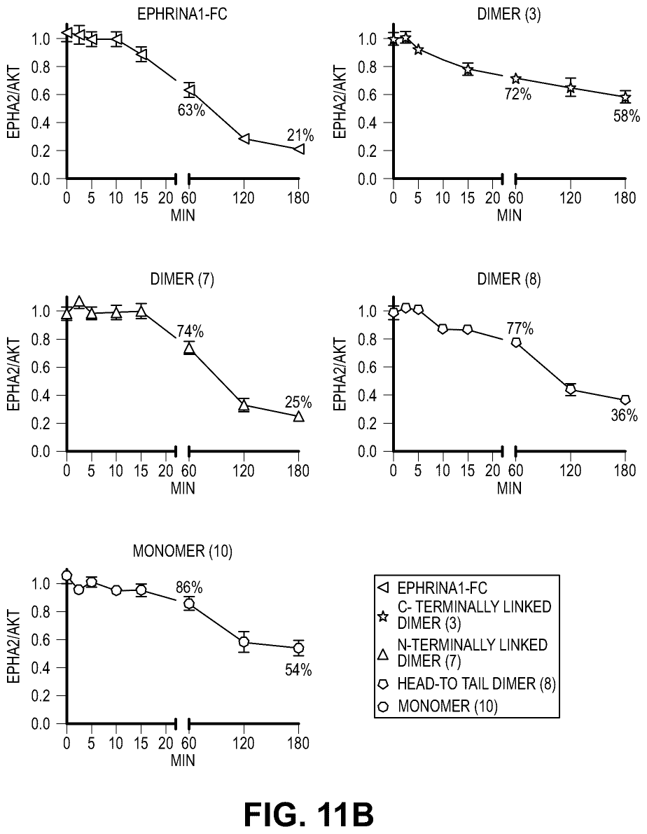

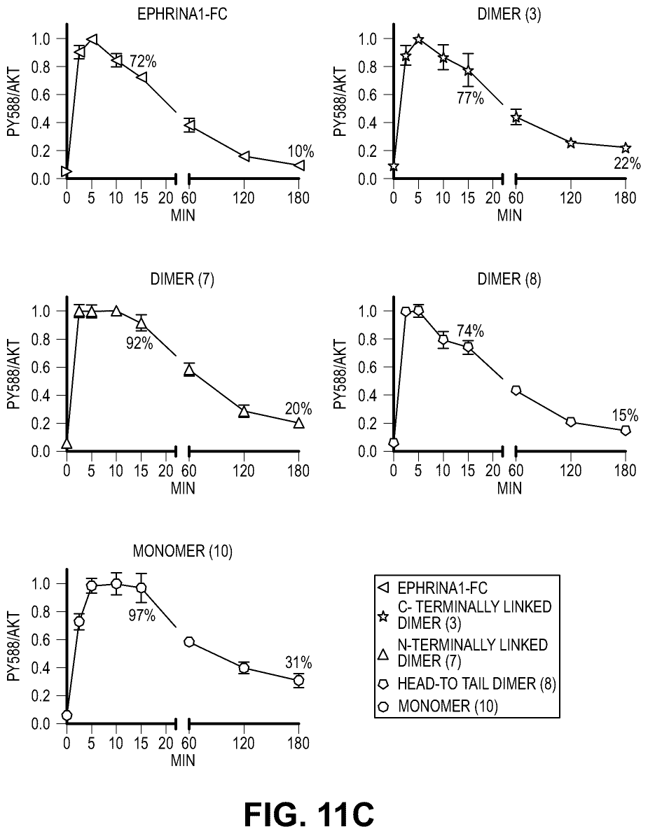

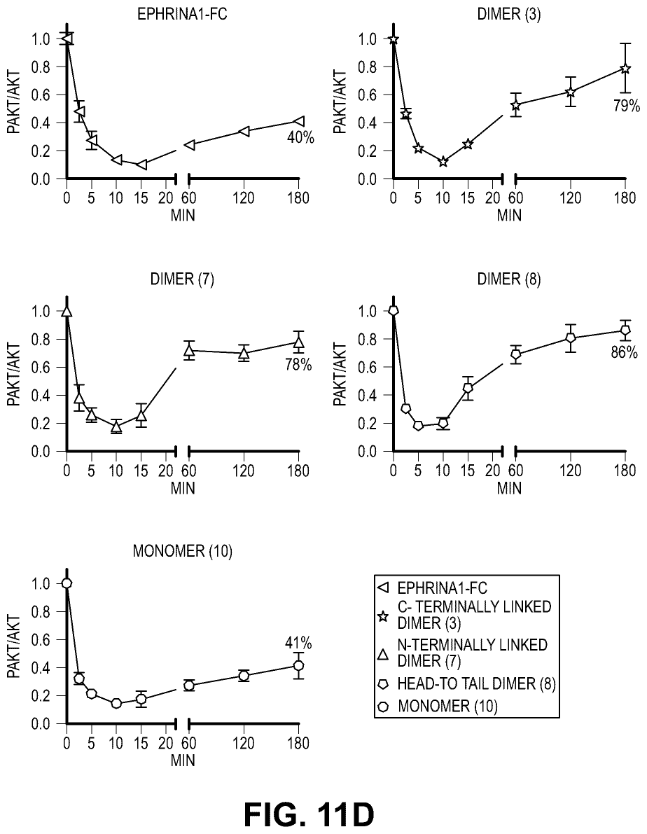

The peak of Y588 phosphorylation normalized to EphA2 (pY588/EphA2) induced by all ligands examined occurred after 2.5-10 min of stimulation and the levels were only slightly reduced after 15 min (FIGS. 3A and 11). This suggests that the configuration of the dimeric peptides does not strongly affect the kinetics of Y588 phosphorylation in the first 15 min of stimulation. Therefore, the C-terminally linked dimers and the monomers are partial agonists that induce lower Eₜₒₚ Y588/EphA2 values. pY588/EphA2 levels gradually decreased after 1 to 3 hours of stimulation, reflecting receptor dephosphorylation, but were still substantially elevated after 3 hours, particularly in the case of the N-terminally linked dimer (7).

EphA2 levels, normalized to AKT as a loading control, decreased after prolonged stimulation (FIG. 3B), as would be expected since ligand-induced EphA2 activation is followed by internalization and degradation with a slower time course (62). EphA2 loss was less pronounced for the C-terminally linked dimer (3) and monomer (10) than for the other ligands, highlighting differences in EphA2 degradation induced by different ligands that may be due to the lower receptor tyrosine phosphorylation levels induced dimer (3) and monomer (10) (FIG. 2C, 2D; Table 3). The amount of EphA2 phosphorylated on Y588 (normalized to AKT as a loading control) persisted at higher levels when induced by dimer (7) and monomer (10) than by the other ligands (FIG. 3C), consistent with the slower receptor dephosphorylation induced by dimer (7) and the slower receptor degradation induced by monomer (10) (FIGS. 3A, 3B). Finally, all dimeric peptides similarly reduced AKT phosphorylation to very low levels, with maximal AKT dephosphorylation observed at ˜10 min (FIG. 3D). AKT phosphorylation then gradually recovered over time, returning to almost the initial level after 3 hours of stimulation in the case of all three dimeric peptides. In contrast, AKT phosphorylation remained low (˜40% of the initial level) after 3 hours of stimulation with ephrinA1-Fc and monomer (10). Thus, saturating concentrations of different ligands have distinctive effects on the time course of EphA2 dephosphorylation and degradation and on the persistence of a downstream signaling effect such as inhibition of AKT.

### Example 8: Dimeric Peptides with Different Configurations Induce EphA2 Oligomers Larger than Dimers

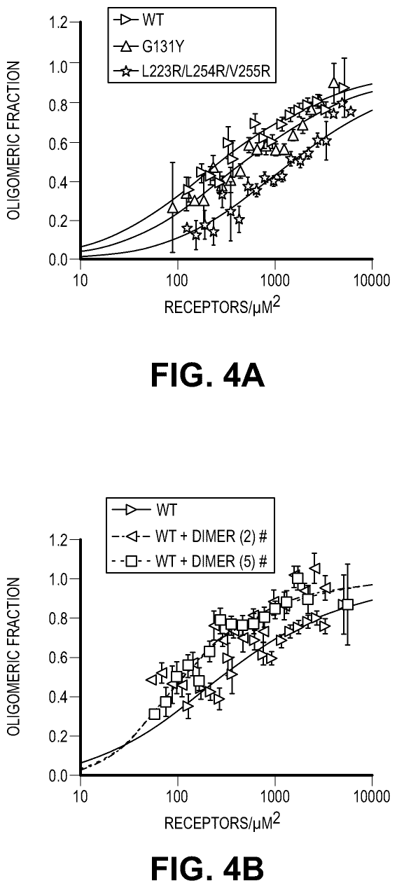

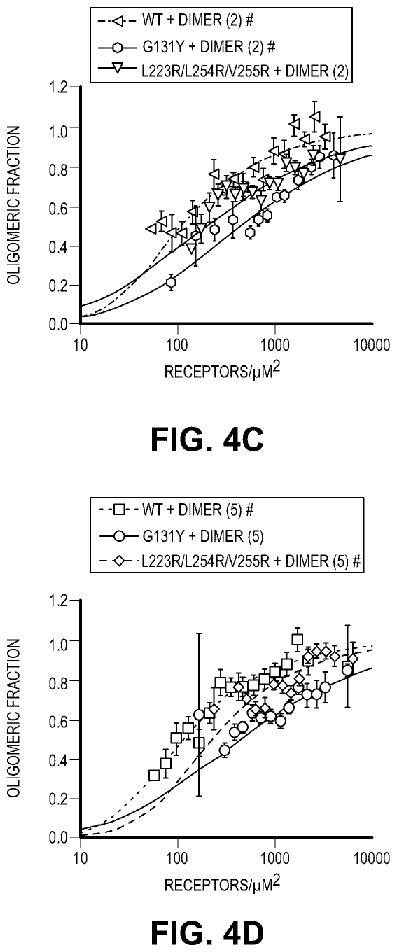

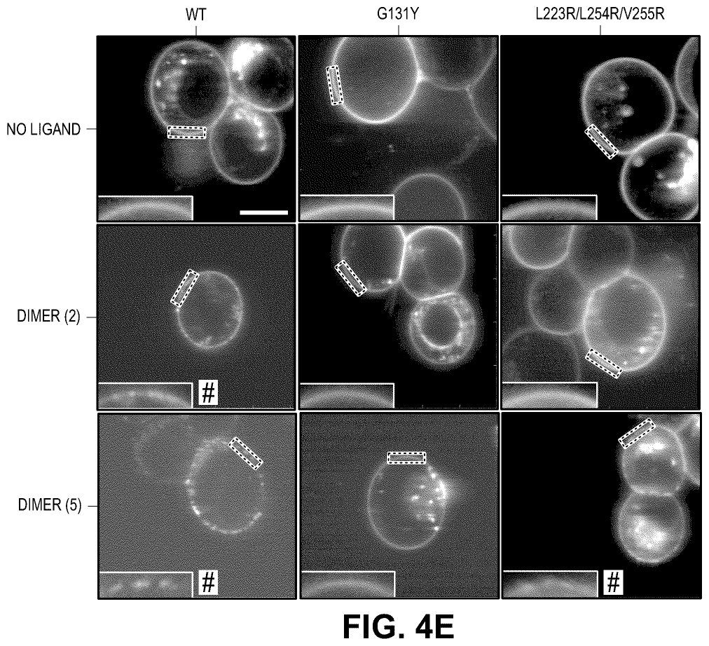

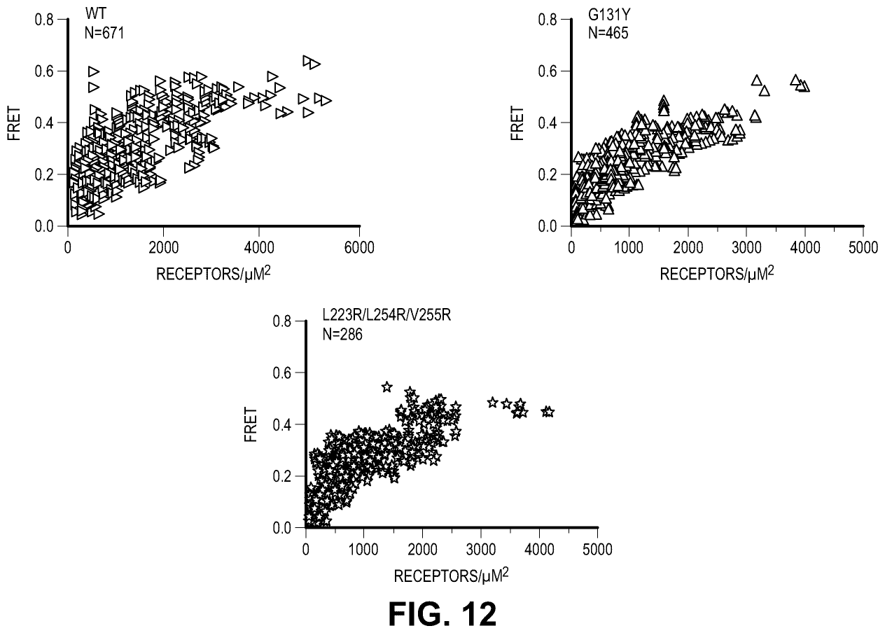

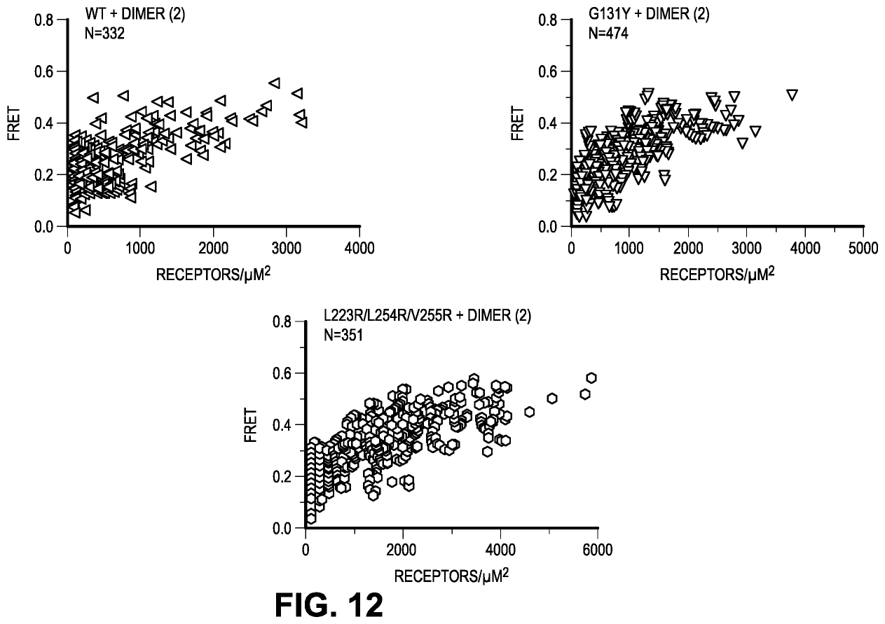

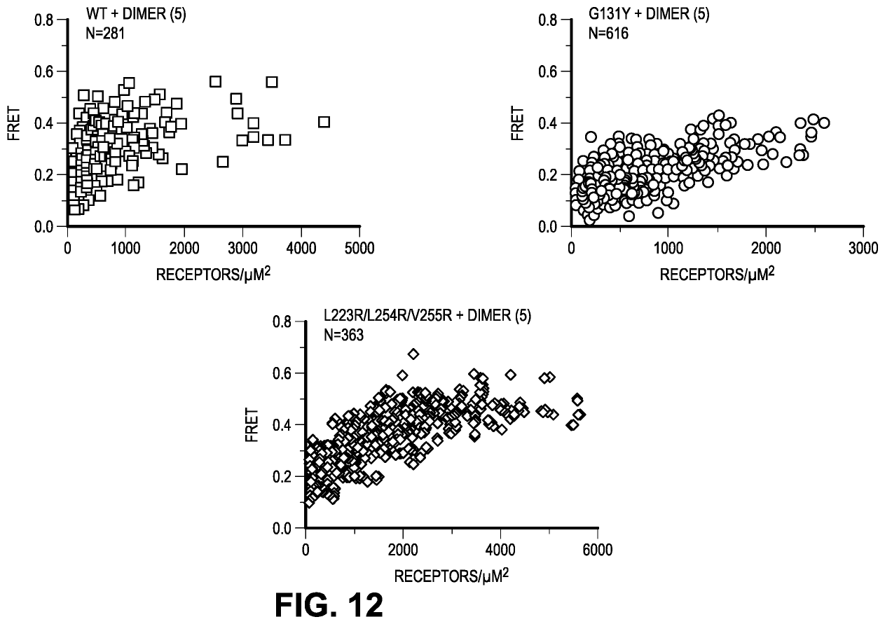

To examine the effects of dimeric peptide ligands on EphA2 oligomerization (including dimerization and higher order clustering), quantitative FRET experiments were performed in live cells. In these experiments, EphA2 molecules tagged at the C-terminus with a donor (mTURQ) or acceptor (EYFP) fluorescent protein are co-expressed in HEK293 cells by transient transfection. FRET is then measured in hundreds of individual cells with different EphA2 expression levels (FIG. 12), and the data are combined to yield average oligomeric fractions at different EphA2 concentrations (FIG. 4A-4D). Oligomerization curves for different monomer-oligomer association models are then fitted to the data points to identify the oligomer model that produces the best fit (i.e. the least mean square error).

In experiments performed in the absence of ligand, EphA2 oligomerization was found to be best described by a monomer-dimer model (FIG. 4A). The dissociation constant determined from fitting the dimerization curve for the EphA2 G131Y mutant, which has impaired ability to assemble through the “dimerization” interface, was similar to that for EphA2 wild-type (WT). In contrast, the dissociation constant determined for the EphA2 L223R/L254R/V255R triple mutant, which has impaired ability to assemble through the previously described “clustering” interface, was significantly higher than for EphA2 WT, indicating that the mutations impair dimerization. These experiments suggested that unliganded EphA2 forms dimers that are stabilized through the clustering interface.

To obtain the data points for oligomerization curves (FIG. 12), the concentration of EphA2 in each small region of plasma membrane in which FRET efficiency was measured. Conversion of fluorescence intensity into accurate 2-dimensional EphA2 concentration requires a reversible hypo-osmotic treatment to swell the cells and smooth the wrinkled topology of their plasma membrane (FIG. 4E). This process does not cause irreversible cell damage or alter membrane protein interactions in a measurable way, and EphA2 is uniformly distributed in the plasma membrane of the swollen cells (FIG. 4E).

To acquire FRET data, the swollen cells were treated with saturating concentrations of the C-terminally linked dimeric peptide (2) and the N-terminally linked dimeric peptide (5). This caused the formation of fluorescent patches of EphA2 WT (FIG. 4E) similar to those observed in response to ephrinA-Fc, which induces EphA2 oligomers that are larger than dimers. Thus, the patches likely reflect EphA2 clustering. The FRET data in the presence of the two peptide ligands are well described by a higher order oligomer model (FIG. 4B, dashed lines), corresponding to steeper oligomerization curves than the dimerization curve for EphA2 WT in the absence of ligand (FIG. 4B, solid line). Interestingly, the oligomerization curves in the presence of the two dimeric peptides are very similar, suggesting that the stabilities of the EphA2 oligomers bound to the two peptides are similar.

The FRET data for the EphA2 G131Y and L223R/L254R/V255R mutants treated with dimeric peptide (2) are best described by a dimerization model (FIG. 4C, solid lines). Consistent with this, these EphA2 mutants do not form patches in the presence of dimer (2) (FIG. 4E). This suggests that mutations in either interface reduce the EphA2 oligomers to dimers. The data for the EphA2 G131Y mutant in the presence of dimeric peptide (5) are also best described by a dimer model (FIG. 4D, solid line), but the data for the EphA2 L223R/L254R/V255R mutant suggest oligomerization (FIG. 4D, dashed line). Consistent with these FRET data, dimer (5) causes patches of the EphA2 L223R/L254R/V255R mutant but not of the G131Y mutant (FIG. 4E). These data suggest that the dimerization interface plays an important role in EphA2 oligomerization in response to dimer (5), while the clustering interface is much less involved. This is consistent with our in silico modeling data which suggests that dimer (5) stabilizes the EphA2 LBD dimer through an interface that is different from the clustering interface (FIGS. 8C and 9).

Taken together, our FRET data are not consistent with a simple EphA2 dimerization model and instead suggest that the dimeric peptides induce larger EphA2 oligomers that utilize different interfaces. The formation of higher order EphA2 oligomers might contribute to the ability of dimeric peptide ligands with different configurations to activate EphA2, by enabling not only cross-phosphorylation within an EphA2 dimer but also phosphorylation by the kinase domain of a neighboring dimer.

### Example 9: A Flexible Juxtamembrane Segment is Required for EphA2 Autophosphorylation

In an EphA2 dimer, the 50 amino acid-long flexible juxtamembrane segment (FIG. 7) could allow an arrangement of the kinase domains suitable for autophosphorylation, independently of the orientation of the LBDs. To investigate the potential involvement of the juxtamembrane segment in EphA2 activation induced by dimeric peptides, stable HEK293 cells expressing EphA2 WT and two EphA2 mutants: the ΔQ565-L582 mutant lacking 18 juxtamembrane residues (Δjxtm-1) and the ΔQ565-T606 mutant lacking 42 residues, which represent most of the juxtamembrane segment (Δjxtm-2) were generated.

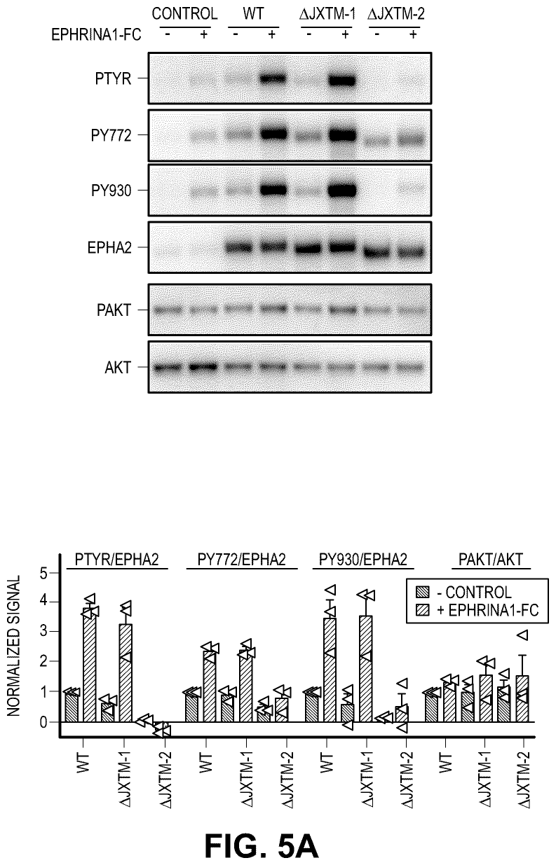

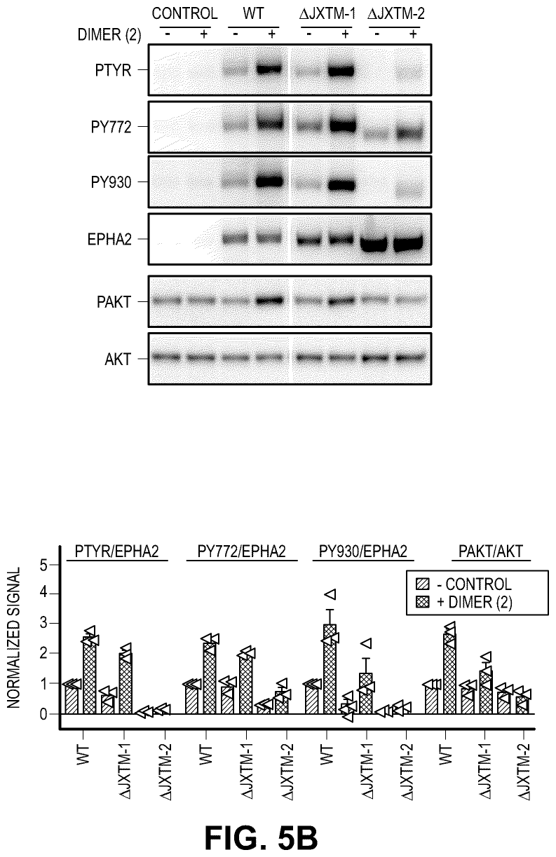

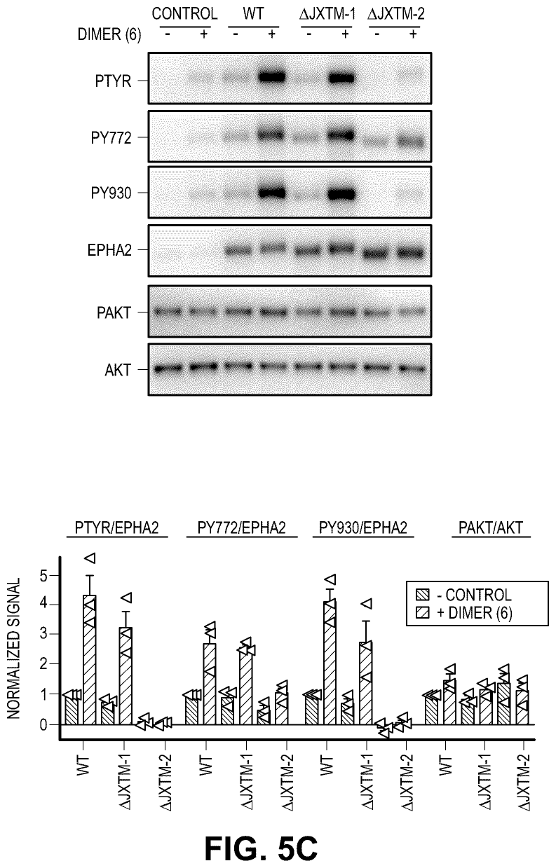

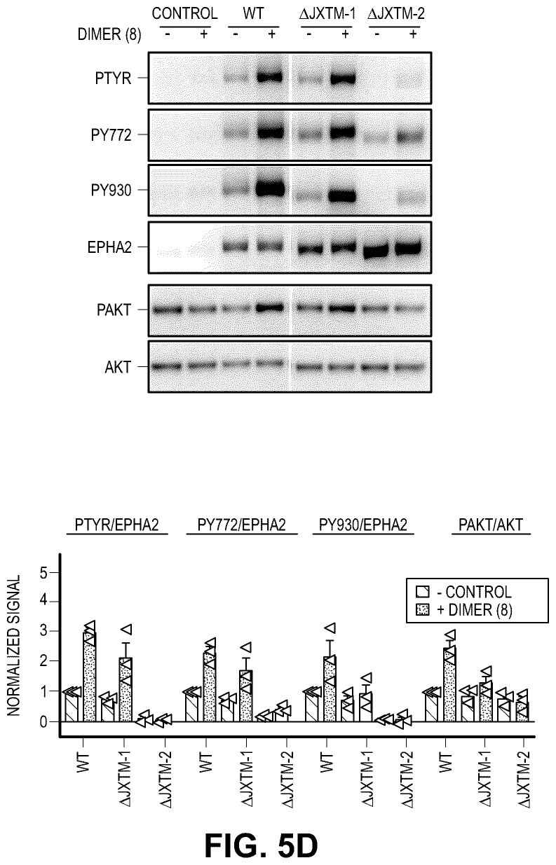

Since the major Y588 and Y594 autophosphorylation sites are in the deleted region of the EphA2 Δjxtm-2 mutant, overall tyrosine phosphorylation as well as phosphorylation of two other major phosphorylation sites still present in the mutants were monitored, Y772 in the activation loop of the kinase domain and Y930 in the SAM domain (FIG. 7). EphA2 WT is substantially tyrosine phosphorylated in the absence of ligand (WT lanes labelled—in the blots in FIGS. 5A-5D), likely because the elevated expression of the transfected EphA2 induces its dimerization. Tyrosine phosphorylation in the absence of ligand was greatly decreased for the EphA2 Δjxtm-2 mutant (FIGS. 5A-5D). Treatment with saturating concentrations of the four dimeric ligands for 2.5 min, to capture the early effects of ligand-induced activation, increased tyrosine phosphorylation of EphA2 WT and the Δjxtm-1 mutant by several folds (FIGS. 5A-5D). Phosphorylation of EphA2 Δjxtm-2 was also in some cases slightly increased, but remained very low. These data suggest that the EphA2 juxtamembrane segment is important to enable appropriate arrangements of EphA2 intracellular regions for cross-phosphorylation on various tyrosine residues both in the absence and in the presence of ligands.

### Example 10: Stimulation with Different Ligands Uncovers EphA2 Biased Signaling

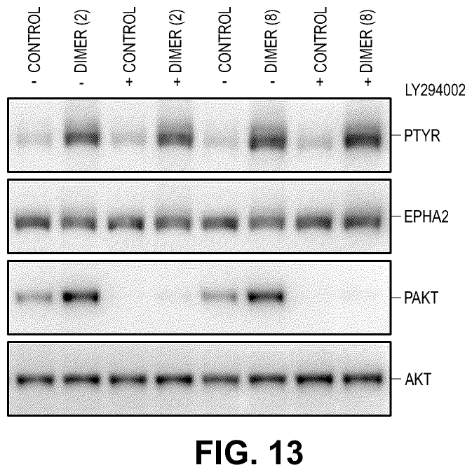

AKT S473 phosphorylation was also assessed in the stably transfected HEK293 cells stimulated for 2.5 min with the four ligands. Unlike the AKT inhibition induced by EphA2 ligands in PC3 cells, in HEK293 cells expressing EphA2 WT an increase in AKT phosphorylation was observed. Peptide dimers (2) and (8) increase AKT phosphorylation more prominently than peptide (6) and ephrinA1-Fc (FIGS. 5A-5D). Furthermore, none of the ligands significantly affected AKT phosphorylation in cells expressing the EphA2 Δjxtm-1 and Δjxtm-2 mutants. Thus, both the EphA2 juxtamembrane segment and the type of arrangement of EphA2 molecules induced by dimers (2) and (8) appear to be important for strong AKT activation by EphA2. Treatment with the PI3-kinase inhibitor LY294002 shows that both basal and EphA2-induced AKT S473 phosphorylation in HEK293 cells depends on PI3-kinase activity (FIG. 13).

Although all four dimeric ligands can similarly activate EphA2 WT, the different effects on AKT phosphorylation of dimers (2) and (8) compared to ephrinA1-Fc and dimer (6) suggest differences in the signaling properties of EphA2 oligomers induced by the different ligands. The observation that two different EphA2 responses (EphA2 tyrosine phosphorylation and AKT phosphorylation) are differentially regulated by distinct ligands suggests ligand functional selectivity or biased signaling, a phenomenon that has been extensively studied for G protein-coupled receptors (GPCRs) but remains poorly documented for receptor tyrosine kinases.

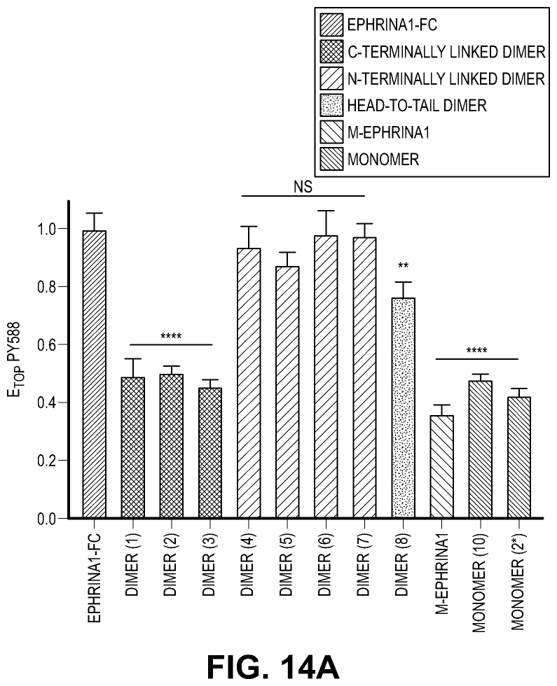

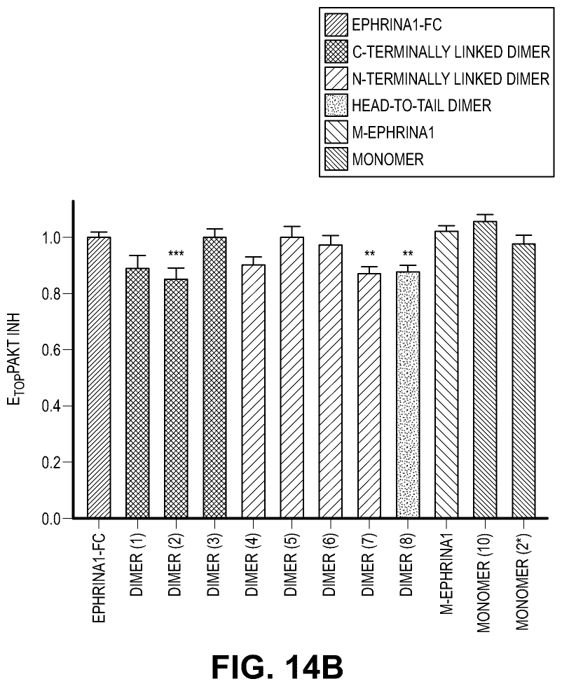

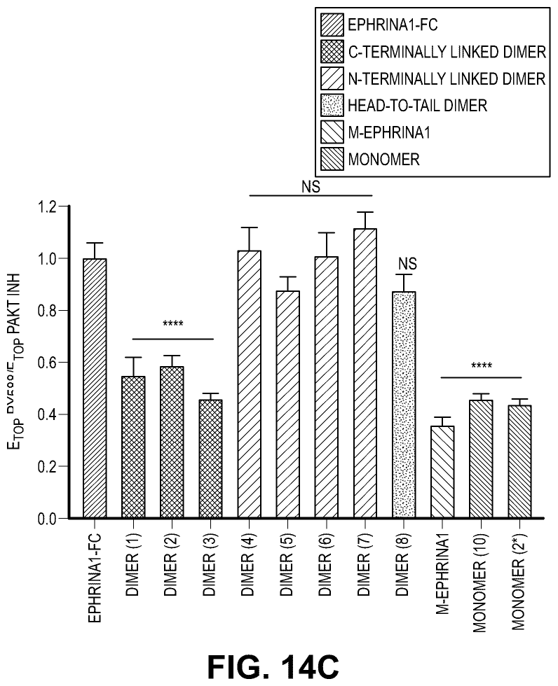

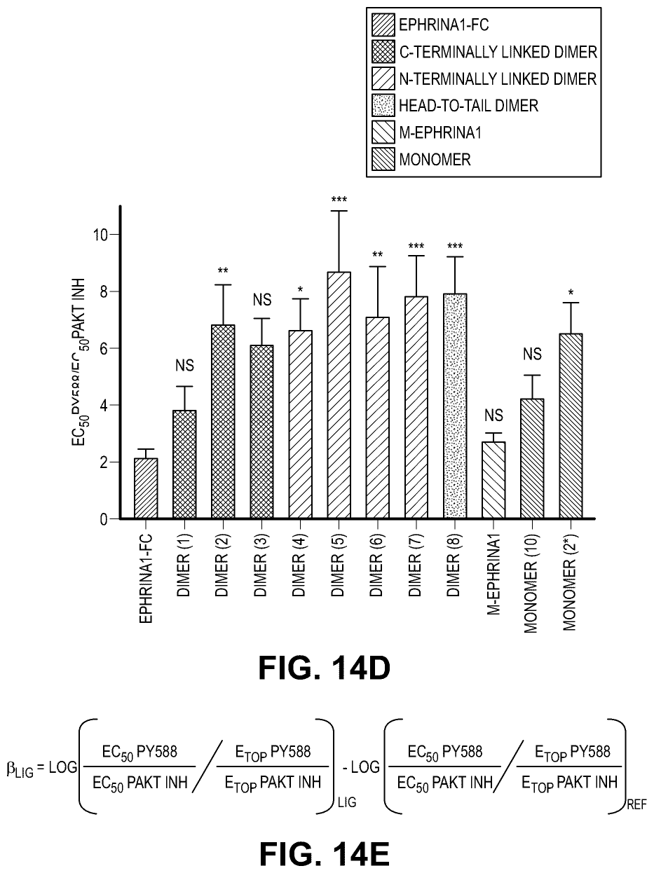

The possibility of EphA2 biased signaling by analyzing the dose-response curves obtained with endogenous EphA2 in PC3 cells (FIG. 2A) was explored using approaches developed for GPCRs. This involves using EphA2 Y588 phosphorylation and AKT phosphorylation quantified as a function of ligand concentration to determine and compare the potency (EC₅₀) and efficacy (Eₜₒₚ) for the two responses induced by different ligands. Remarkably, large differences among the ligands in the Eₜₒₚ values for Y588 phosphorylation (FIGS. 2C, 2D and 14A), but not in the Eₜₒₚ values for AKT inhibition (FIGS. 2E and 14B), were observed. In terms of relative efficacies for pY588 and pAKT inhibition, the C-terminally linked dimers and monomeric ligands behave differently from ephrinA1-Fc, while the N-terminally linked dimers and the head-to-tail dimer (8) are similar to ephrinA1-Fc (FIG. 14C). Unlike the Eₜₒₚ values, when comparing the relative potency (EC₅₀) values for pY588 and pAKT inhibition, the N-terminally linked dimers and head-to-tail dimer (8) are all significantly different from ephrinA1-Fc (FIG. 13D). Thus, the type of linkage affects EphA2 signaling properties induced by the dimeric peptides.

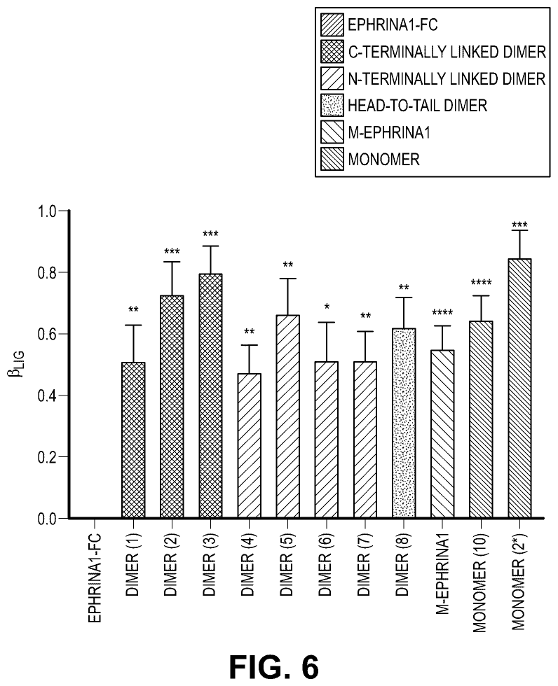

The determined EC₅₀ and Eₜₒₚ values allowed us to calculate the bias factor β_{lig} for the two different responses induced by the various ligands relative to ephrinA1-Fc as the reference ligand (FIG. 14E). This revealed that all the peptides tested are biased ligands compared to ephrinA1-Fc and that they bias EphA2 signaling towards AKT inhibition relative to Y588 phosphorylation (FIG. 6; Table 3). Remarkably, the bias originates from different mechanisms that depend on the class of ligands, with the N-terminally linked and head-to-tail dimers modulating relative potencies and the C-terminally-linked dimers and monomers modulating relative efficacies.

1-105. (canceled)

106. A composition comprising a peptide comprising at least a first subunit and a second subunit, wherein said first subunit comprises X1-W-L-A-Y-P-D-S-V-P-Y-X2, and wherein said second subunit comprises X1-W-L-A-Y-P-D-S-V-P-Y-X2, wherein
X1 is A, βAlanine, a first spacer, C, azido-lysine (K_{N3}), propargylglycine (Pra), or any combination thereof; and
X2 is R, a second spacer, P-K, C, K, or any combination thereof.

107. The composition of claim 106, wherein said C is carbamidomethyl-cysteine (C_{cam}).

108. The composition of claim 106, wherein a C-terminus of said first subset, said second subset, or both is amidated.

109. The composition of claim 106, wherein said first spacer and said second spacer comprise one or more amino acids.

110. The composition of claim 106, wherein said first spacer and said second spacer comprise a glycine.

111. The composition of claim 106, wherein said first spacer and said second spacer comprise a glycine and a serine.

112. The composition of claim 106, wherein said first subunit and said second subunit are homologous.

113. The composition of claim 106, wherein an N-terminus of said first subunit or said second subunit, or a C-terminus of said first subunit or said second subunit, or any combination thereof, comprises biotin.

114. The composition of claim 106, wherein said first subunit or said second subunit further comprises acetylation of a Lys14 side chain.

115. The composition of claim 106, wherein said composition comprises any one of the peptides represented by SEQ ID NOs: 1-8.

116. A pharmaceutical composition comprising
the composition of claim 106; and
one or more pharmaceutically acceptable excipients.

117. A method of treating a disease or condition in a subject in need thereof, comprising administering the pharmaceutical composition of claim 116, to said subject.

118. The method of claim 117, further comprising administering a half-life extending molecule to said subject.

119. The method of claim 117, wherein said disease or condition is a parasitic infection.

120. The method of claim 117, wherein said disease or condition is pathological forms of angiogenesis.

121. The method of claim 117, wherein said disease or condition is an inflammatory disease.

122. The method of claim 121, wherein said inflammatory disease is atherosclerosis, diabetes, arthritis, psoriasis, multiple sclerosis, lupus, inflammatory bowel disease, Addison's disease, Grave's disease, Sjogren's syndrome, Hashimoto's thyroiditis, Myasthenia gravis, Autoimmune vasculitis, Pernicious anemia, graft-versus-host disease, or Celiac disease.

123. The method of claim 117, wherein said disease or condition is cancer.

124. The method of claim 123, wherein said cancer is prostate cancer, castration resistant prostate cancer, neuroendocrine prostate cancer, transitional cell prostate cancer, squamous cell prostate cancer, or small cell prostate cancer.

125. A method of preventing or reversing the onset of a subset of a disease or condition in a subject suffering from a disease or condition, comprising administering the pharmaceutical composition of claim 116 to said subject.
